SEQUENCE LISTING (62 sequences)

SEQ ID NO: 1 - protein, 454 aa, synthetic construct
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 2 - protein, 238 aa, synthetic construct
DLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFS

SEQ ID NO: 3 - DNA, 354 bp, synthetic construct
  source 1..354: mol_type = other DNA
cagatccagctgcagcagtctggacctgagctggtgaagcctggggcttcagtgaagctgtcctgcaagacttctggcttcaccttcagcagtagttatataagttggttgaagcaaaagcctggacagagtcttgagtggattgcatggatttatgctggaactggaaatactaactataatcagaagttcacaggcaaggcccaactgactgtagacacatcctccagcacagcctacatgcaattcagcagcctgacaactgaggactctgccatctattactgtgcaagaccgggagagggttcaccctttgcttactggggccaagggactctggtcactgtctctgca

SEQ ID NO: 4 - protein, 118 aa, synthetic construct
QIQLQQSGPELVKPGASVKLSCKTSGFTFSSSYISWLKQKPGQSLEWIAWIYAGTGNTNYNQKFTGKAQLTVDTSSSTAYMQFSSLTTEDSAIYYCARPGEGSPFAYWGQGTLVTVSA

SEQ ID NO: 5 - DNA, 321 bp, synthetic construct
  source 1..321: mol_type = other DNA
gatatccagatgacacagactacatcctccctgtccgcctctctgggagacagagtcaccatcagttgcagggcaagtcaggacattagcaattatttaaattggtatcagcagaaaccagatggaactgttaaagtcctgatttactacacatcaagattatactcaggagtcccatcaaggttcagtggcagtgggtctggaacagattattctctcaccattagcaacctggagcaagaagatattgccacttacttttgccaacagggtgatacgcttccgtacacgttcggaggggggaccaagctggaaataaaa

SEQ ID NO: 6 - protein, 107 aa, synthetic construct
DIQMTQTTSSLSASLGDRVTISCRASQDISNYLNWYQQKPDGTVKVLIYYTSRLYSGVPSRFSGSGSGTDYSLTISNLEQEDIATYFCQQGDTLPYTFGGGTKLEIK

SEQ ID NO: 7 - DNA, 363 bp, synthetic construct
  source 1..363: mol_type = other DNA
gacgtgaagctcgtggagtctgggggaggcttagtgaagcttggagggtccctgaaactctcctgtgcagcctctggattcactttcagtagccattacatgtcttgggttcgccagactccagagaagaggctggagttggtcgctgccattaataataaggatggtatcacctactatccagacactgtgaagggccgactcaccatctccagagacattgccaagaacaccctgtacctgcaaatgagcagtctgaggtctgaggacacagccttgtattactgtacaagacaacctagtagcccctattactatgctatggactactggggtcaaggaacctcagtcaccgtctcctca

SEQ ID NO: 8 - protein, 121 aa, synthetic construct
DVKLVESGGGLVKLGGSLKLSCAASGFTFSSHYMSWVRQTPEKRLELVAAINNKDGITYYPDTVKGRLTISRDIAKNTLYLQMSSLRSEDTALYYCTRQPSSPYYYAMDYWGQGTSVTVSS

SEQ ID NO: 9 - DNA, 333 bp, synthetic construct
  source 1..333: mol_type = other DNA
gacattgtgctgacccagtctccagcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctccgacgttcggtggaggcaccaagctggaaatcaaa

SEQ ID NO: 10 - protein, 111 aa, synthetic construct
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPPTFGGGTKLEIK

SEQ ID NO: 11 - protein, 5 aa, synthetic construct
SSYIS

SEQ ID NO: 12 - protein, 8 aa, synthetic construct
IYAGTGNT

SEQ ID NO: 13 - protein, 9 aa, synthetic construct
PGEGSPFAY

SEQ ID NO: 14 - protein, 6 aa, synthetic construct
QDISNY

SEQ ID NO: 15 - protein, 7 aa, synthetic construct
YTSRLYS

SEQ ID NO: 16 - protein, 9 aa, synthetic construct
QQGDTLPYT

SEQ ID NO: 17 - protein, 5 aa, synthetic construct
SHYMS

SEQ ID NO: 18 - protein, 8 aa, synthetic construct
INNKDGIT

SEQ ID NO: 19 - protein, 12 aa, synthetic construct
QPSSPYYYAMDY

SEQ ID NO: 20 - protein, 10 aa, synthetic construct
KSVSTSGYSY

SEQ ID NO: 21 - protein, 7 aa, synthetic construct
LASNLES

SEQ ID NO: 22 - protein, 9 aa, synthetic construct
QHSRELPPT

SEQ ID NO: 23 - DNA, 353 bp, synthetic construct
  source 1..353: mol_type = other DNA
aaattcagctgcagcagagcggccccgagctggtgaagcccggcgctagcgtgaagctgagctgcaagacaagcggcttcaccttcagcagcagctacatcagctggctgaagcagaagcccgggcagagcctggagtggatcgcctggatctacgccggcaccggcaacaccaactacaatcagaagttcaccggcaaggctcagctgaccgtggacacaagcagcagcaccgcctacatgcagttcagcagcctgaccaccgaggacagcgccatctactactgcgctagacccggcgagggcagccccttcgcctactggggccaaggcaccctggtgaccgtgagcgcc

SEQ ID NO: 24 - DNA, 321 bp, synthetic construct
  source 1..321: mol_type = other DNA
gacattcagatgacacagaccacaagcagcctgagcgctagcctgggcgacagagtgaccatcagctgcagagctagccaagacatcagcaactacctgaactggtatcagcagaagcccgacgggaccgtgaaggtgctgatctactacacaagcagactgtacagcggcgtgcctagcagattcagcggcagcggcagcggcaccgactacagcctgaccatcagcaacctggagcaagaggacatcgccacctacttctgtcagcaaggcgacaccctgccctacaccttcggcgggggcaccaagctggagatcaag

SEQ ID NO: 25 - DNA, 363 bp, synthetic construct
  source 1..363: mol_type = other DNA
gacgtcaagctcgtggaatccggcgggggcctcgtgaagctgggcggcagcctgaagctgagctgcgccgctagcggcttcaccttcagcagccactacatgagctgggtgagacagacccccgagaagagactggagctggtggccgccatcaacaacaaggacggcatcacctactaccccgacaccgtgaagggcagactgaccatcagcagagacatcgccaagaacaccctgtacctgcagatgagcagcctgagaagcgaggacaccgccctgtactactgcacaagacagcctagcagcccctactattacgccatggactactggggccaaggcacaagcgtgaccgtgagcagc

SEQ ID NO: 26 - DNA, 333 bp, synthetic construct
  source 1..333: mol_type = other DNA
gacatcgtgctgacacagagccccgctagcctggccgtgagcctggggcagagagccaccatcagctgcagagctagcaagagcgtgagcacaagcggctacagctacatgcactggtatcagcagaagcccgggcagccccccaagctgctgatctacctggctagcaacctggagagcggcgtgcccgctagattcagcggcagcggcagcggcaccgacttcaccctgaacatccaccccgtggaggaagaggacgccgccacctactactgtcagcacagcagagagctgccccccacattcggcgggggcacaaagctcgaaatcaag

SEQ ID NO: 27 - DNA, 45 bp, synthetic construct
  source 1..45: mol_type = other DNA
ggtggcggtggcagcggcggcggtggtagcggtggaggcggctca

SEQ ID NO: 28 - protein, 608 aa, synthetic construct
MALPVTALLLPLALLLHAARPGSGDIQMTQTTSSLSASLGDRVTISCRASQDISNYLNWYQQKPDGTVKVLIYYTSRLYSGVPSRFSGSGSGTDYSLTISNLEQEDIATYFCQQGDTLPYTFGGGTKLEIKGGGGSGGGGSGGGGSGGGGSQIQLQQSGPELVKPGASVKLSCKTSGFTFSSSYISWLKQKPGQSLEWIAWIYAGTGNTNYNQKFTGKAQLTVDTSSSTAYMQFSSLTTEDSAIYYCARPGEGSPFAYWGQGTLVTVSAASSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLHMQALPPRLEGGGEGRGSLLTCGDVEENPGPRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 29 - DNA, 1830 bp, synthetic construct
  source 1..1830: mol_type = other DNA
gccaccatggccctccctgtcaccgccctgctgcttccgctggctcttctgctccacgccgctcggcccggatccggagacattcagatgacacagaccacaagcagcctgagcgctagcctgggcgacagagtgaccatcagctgcagagctagccaagacatcagcaactacctgaactggtatcagcagaagcccgacgggaccgtgaaggtgctgatctactacacaagcagactgtacagcggcgtgcctagcagattcagcggcagcggcagcggcaccgactacagcctgaccatcagcaacctggagcaagaggacatcgccacctacttctgtcagcaaggcgacaccctgccctacaccttcggcgggggcaccaagctggagatcaagggcgggggcggcagcggcgggggcgggagcggcgggggcgggagcgggggcggcgggagccaaattcagctgcagcagagcggccccgagctggtgaagcccggcgctagcgtgaagctgagctgcaagacaagcggcttcaccttcagcagcagctacatcagctggctgaagcagaagcccgggcagagcctggagtggatcgcctggatctacgccggcaccggcaacaccaactacaatcagaagttcaccggcaaggctcagctgaccgtggacacaagcagcagcaccgcctacatgcagttcagcagcctgaccaccgaggacagcgccatctactactgcgctagacccggcgagggcagccccttcgcctactggggccaaggcaccctggtgaccgtgagcgccgctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttgcacatgcaggccctgccccctcgcctcgagggcggcggagagggcagaggaagtcttctaacatgcggtgacgtggaggagaatcccggccctaggatgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtc

SEQ ID NO: 30 - protein, 677 aa, synthetic construct
MALPVTALLLPLALLLHAARPGSGDIQMTQTTSSLSASLGDRVTISCRASQDISNYLNWYQQKPDGTVKVLIYYTSRLYSGVPSRFSGSGSGTDYSLTISNLEQEDIATYFCQQGDTLPYTFGGGTKLEIKGGGGSGGGGSGGGGSGGGGSQIQLQQSGPELVKPGASVKLSCKTSGFTFSSSYISWLKQKPGQSLEWIAWIYAGTGNTNYNQKFTGKAQLTVDTSSSTAYMQFSSLTTEDSAIYYCARPGEGSPFAYWGQGTLVTVSAASSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 31 - DNA, 2037 bp, synthetic construct
  source 1..2037: mol_type = other DNA
gccaccatggccctccctgtcaccgccctgctgcttccgctggctcttctgctccacgccgctcggcccggatccggagacattcagatgacacagaccacaagcagcctgagcgctagcctgggcgacagagtgaccatcagctgcagagctagccaagacatcagcaactacctgaactggtatcagcagaagcccgacgggaccgtgaaggtgctgatctactacacaagcagactgtacagcggcgtgcctagcagattcagcggcagcggcagcggcaccgactacagcctgaccatcagcaacctggagcaagaggacatcgccacctacttctgtcagcaaggcgacaccctgccctacaccttcggcgggggcaccaagctggagatcaagggcgggggcggcagcggcgggggcgggagcggcgggggcgggagcgggggcggcgggagccaaattcagctgcagcagagcggccccgagctggtgaagcccggcgctagcgtgaagctgagctgcaagacaagcggcttcaccttcagcagcagctacatcagctggctgaagcagaagcccgggcagagcctggagtggatcgcctggatctacgccggcaccggcaacaccaactacaatcagaagttcaccggcaaggctcagctgaccgtggacacaagcagcagcaccgcctacatgcagttcagcagcctgaccaccgaggacagcgccatctactactgcgctagacccggcgagggcagccccttcgcctactggggccaaggcaccctggtgaccgtgagcgccgctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctcgagggcggcggagagggcagaggaagtcttctaacatgcggtgacgtggaggagaatcccggccctaggatgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtc

SEQ ID NO: 32 - protein, 924 aa, synthetic construct
MALPVTALLLPLALLLHAARPGSGDIQMTQTTSSLSASLGDRVTISCRASQDISNYLNWYQQKPDGTVKVLIYYTSRLYSGVPSRFSGSGSGTDYSLTISNLEQEDIATYFCQQGDTLPYTFGGGTKLEIKGGGGSGGGGSGGGGSGGGGSQIQLQQSGPELVKPGASVKLSCKTSGFTFSSSYISWLKQKPGQSLEWIAWIYAGTGNTNYNQKFTGKAQLTVDTSSSTAYMQFSSLTTEDSAIYYCARPGEGSPFAYWGQGTLVTVSAASSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMTRLTVLALLAGLLASSRAEVQLVESGGGLVQPGVSLRLSCAASGFTFTSYTMSWVRQTPGKGLEWVAFISGGGGDTYYPDSVKGRFTISRDNSKNTLYLQMNSLRAEDTAVYYCARHGYDGTWFAYWGQGTLVTVSSGGGGSGGGGSGGGGSDVLTQSPSSLSASVGDRVTITCRASENIYSYLAWYQQKPGKSPKLLVSNAKTLAEGVPSRFSGSGSGTQFSLTISSLQPEDFATYYCQQHYATPYTFGGGTKLEIKRTVGGGGSGGGGSGGGGSQGQDRHMIRMRQLIDCVDQLKNYVNDLVPEFLPAPEDVETNCEWSAFSCFQKAQLKSANTGNNERIINVCIKKLKRNLWGLAGLNSCPSCDSYEKKPPKEFLERFKSLLQKMIHQHLSSRTHGSEDS

SEQ ID NO: 33 - DNA, 2778 bp, synthetic construct
  source 1..2778: mol_type = other DNA
gccaccatggccctccctgtcaccgccctgctgcttccgctggctcttctgctccacgccgctcggcccggatccggagacattcagatgacacagaccacaagcagcctgagcgctagcctgggcgacagagtgaccatcagctgcagagctagccaagacatcagcaactacctgaactggtatcagcagaagcccgacgggaccgtgaaggtgctgatctactacacaagcagactgtacagcggcgtgcctagcagattcagcggcagcggcagcggcaccgactacagcctgaccatcagcaacctggagcaagaggacatcgccacctacttctgtcagcaaggcgacaccctgccctacaccttcggcgggggcaccaagctggagatcaagggcgggggcggcagcggcgggggcgggagcggcgggggcgggagcgggggcggcgggagccaaattcagctgcagcagagcggccccgagctggtgaagcccggcgctagcgtgaagctgagctgcaagacaagcggcttcaccttcagcagcagctacatcagctggctgaagcagaagcccgggcagagcctggagtggatcgcctggatctacgccggcaccggcaacaccaactacaatcagaagttcaccggcaaggctcagctgaccgtggacacaagcagcagcaccgcctacatgcagttcagcagcctgaccaccgaggacagcgccatctactactgcgctagacccggcgagggcagccccttcgcctactggggccaaggcaccctggtgaccgtgagcgccgctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctcgagggcggcggagagggcagaggaagtcttctaacatgcggtgacgtggaggagaatcccggccctaggatgaccaggctgacagtgctggccctgctggcaggactgctggcaagctccagggccgaggtgcagctggtggagtccggcggcggcctggtgcagcctggagtatccctgaggctgtcttgcgcagcaagcggcttcacctttacatcctacaccatgtcttgggtgagacagaccccaggcaagggactggagtgggtggccttcatcagcggcggaggaggcgacacatactatcctgattccgtgaagggccggtttaccatcagcagagacaactccaagaatacactgtatctgcagatgaactccctgagggcagaggacaccgccgtgtactattgcgccagacacggctacgatggcacatggttcgcctattggggccagggcaccctggtgacagtgtctagcggaggaggaggatctggaggaggaggaagcggaggaggaggatccgacgtgctgacccagagcccatcctctctgtctgccagcgtgggcgatagggtgaccatcacatgtcgcgcctctgagaacatctacagctatctggcctggtaccagcagaagcccggcaagtcccctaagctgctggtgtctaatgcaaagaccctggcagagggagtgccatctaggttctccggctctggcagcggcacccagtttagcctgacaatcagctccctgcagcctgaggatttcgccacatactattgtcagcagcactacgccaccccatatacatttggcggcggcaccaagctggagatcaagaggacagtgggaggaggaggaagcggaggaggaggatccggcggcggcggctctcaaggtcaagatcgccacatgattagaatgcgtcaacttatagattgtgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatgtattaaaaagctgaagaggaacctctggggcctggcgggcttgaattcctgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 34 - protein, 615 aa, synthetic construct
MALPVTALLLPLALLLHAARPGSGDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPPTFGGGTKLEIKGGGGSGGGGSGGGGSGGGGSDVKLVESGGGLVKLGGSLKLSCAASGFTFSSHYMSWVRQTPEKRLELVAAINNKDGITYYPDTVKGRLTISRDIAKNTLYLQMSSLRSEDTALYYCTRQPSSPYYYAMDYWGQGTSVTVSSASSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLHMQALPPRLEGGGEGRGSLLTCGDVEENPGPRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 35 - DNA, 1851 bp, synthetic construct
  source 1..1851: mol_type = other DNA
gccaccatggccctccctgtcaccgccctgctgcttccgctggctcttctgctccacgccgctcggcccggatccggagacatcgtgctgacacagagccccgctagcctggccgtgagcctggggcagagagccaccatcagctgcagagctagcaagagcgtgagcacaagcggctacagctacatgcactggtatcagcagaagcccgggcagccccccaagctgctgatctacctggctagcaacctggagagcggcgtgcccgctagattcagcggcagcggcagcggcaccgacttcaccctgaacatccaccccgtggaggaagaggacgccgccacctactactgtcagcacagcagagagctgccccccacattcggcgggggcacaaagctcgaaatcaagggcgggggcggcagcggcgggggcgggagcggcgggggcgggagcgggggcggcgggagcgacgtcaagctcgtggaatccggcgggggcctcgtgaagctgggcggcagcctgaagctgagctgcgccgctagcggcttcaccttcagcagccactacatgagctgggtgagacagacccccgagaagagactggagctggtggccgccatcaacaacaaggacggcatcacctactaccccgacaccgtgaagggcagactgaccatcagcagagacatcgccaagaacaccctgtacctgcagatgagcagcctgagaagcgaggacaccgccctgtactactgcacaagacagcctagcagcccctactattacgccatggactactggggccaaggcacaagcgtgaccgtgagcagcgctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttgcacatgcaggccctgccccctcgcctcgagggcggcggagagggcagaggaagtcttctaacatgcggtgacgtggaggagaatcccggccctaggatgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtc

SEQ ID NO: 36 - skipped

SEQ ID NO: 37 - DNA, 63 bp, synthetic construct
  source 1..63: mol_type = other DNA
atggccctccctgtcaccgccctgctgcttccgctggctcttctgctccacgccgctcggccc

SEQ ID NO: 38 - protein, 21 aa, synthetic construct
MALPVTALLLPLALLLHAARP

SEQ ID NO: 39 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 40 - protein, 45 aa, synthetic construct
TTTPAPRPPTPAPTIASQPLSLRPEACRPAAGGAVHTRGLDFACD

SEQ ID NO: 41 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 42 - protein, 72 aa, synthetic construct
ILETYRILETRPALAPROLEUALAGLYTHRCYSGLYVALLEULEULEUSERLEUVALILETHRLEUTYRCYS

SEQ ID NO: 43 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 44 - protein, 42 aa, synthetic construct
KRGRKKLLYIFKQPFMRPVQTTQEEDGCSCRFPEEEEGGCEL

SEQ ID NO: 45 - DNA, 129 bp, synthetic construct
  source 1..129: mol_type = other DNA
agagtgaagttcagcaggagcgcagacgcccccgcgtacaagcagggccagaaccagctctataacgagctcaatctaggacgaagagaggagtacgatgttttgcacatgcaggccctgccccctcgc

SEQ ID NO: 46 - protein, 43 aa, synthetic construct
RVKFSRSADAPAYKQGQNQLYNELNLGRREEYDVLHMQALPPR

SEQ ID NO: 47 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 48 - protein, 79 aa, synthetic construct
RVKFSRSADAPAYKQGQNQLYNELNLGRREEYDVLDKRRGRDPEMGGKPRRKNPQEGLYNELQKDKMAEAYSEIGMKGE

SEQ ID NO: 49 - DNA, 54 bp, synthetic construct
  source 1..54: mol_type = other DNA
gagggcagaggaagtcttctaacatgcggtgacgtggaggagaatcccggccct

SEQ ID NO: 50 - protein, 18 aa, synthetic construct
EGRGSLLTCGDVEENPGP

SEQ ID NO: 51 - DNA, 474 bp, synthetic construct
  source 1..474: mol_type = other DNA
atgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtctga

SEQ ID NO: 52 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 53 - DNA, 27 bp, synthetic construct
  source 1..27: mol_type = other DNA
tgtccttacagcaacccaagcctctgc

SEQ ID NO: 54 - protein, 9 aa, synthetic construct
CPYSNPSLC

SEQ ID NO: 55 - DNA, 786 bp, synthetic construct
  source 1..786: mol_type = other DNA
atgaccaggctgacagtgctggccctgctggcaggactgctggcaagctccagggccgaggtgcagctggtggagtccggcggcggcctggtgcagcctggagtatccctgaggctgtcttgcgcagcaagcggcttcacctttacatcctacaccatgtcttgggtgagacagaccccaggcaagggactggagtgggtggccttcatcagcggcggaggaggcgacacatactatcctgattccgtgaagggccggtttaccatcagcagagacaactccaagaatacactgtatctgcagatgaactccctgagggcagaggacaccgccgtgtactattgcgccagacacggctacgatggcacatggttcgcctattggggccagggcaccctggtgacagtgtctagcggaggaggaggatctggaggaggaggaagcggaggaggaggatccgacgtgctgacccagagcccatcctctctgtctgccagcgtgggcgatagggtgaccatcacatgtcgcgcctctgagaacatctacagctatctggcctggtaccagcagaagcccggcaagtcccctaagctgctggtgtctaatgcaaagaccctggcagagggagtgccatctaggttctccggctctggcagcggcacccagtttagcctgacaatcagctccctgcagcctgaggatttcgccacatactattgtcagcagcactacgccaccccatatacatttggcggcggcaccaagctggagatcaagaggacagtg

SEQ ID NO: 56 - protein, 262 aa, synthetic construct
MTRLTVLALLAGLLASSRAEVQLVESGGGLVQPGVSLRLSCAASGFTFTSYTMSWVRQTPGKGLEWVAFISGGGGDTYYPDSVKGRFTISRDNSKNTLYLQMNSLRAEDTAVYYCARHGYDGTWFAYWGQGTLVTVSSGGGGSGGGGSGGGGSDVLTQSPSSLSASVGDRVTITCRASENIYSYLAWYQQKPGKSPKLLVSNAKTLAEGVPSRFSGSGSGTQFSLTISSLQPEDFATYYCQQHYATPYTFGGGTKLEIKRTV

SEQ ID NO: 57 - DNA, 45 bp, synthetic construct
  source 1..45: mol_type = other DNA
ggaggaggaggaagcggaggaggaggatccggcggcggcggctct

SEQ ID NO: 58 - DNA, 381 bp, synthetic construct
  source 1..381: mol_type = other DNA
caaggtcaagatcgccacatgattagaatgcgtcaacttatagattgtgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatgtattaaaaagctgaagaggaacctctggggcctggcgggcttgaattcctgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 59 - protein, 404 aa, synthetic construct
MTRLTVLALLAGLLASSRAEVQLVESGGGLVQPGVSLRLSCAASGFTFTSYTMSWVRQTPGKGLEWVAFISGGGGDTYYPDSVKGRFTISRDNSKNTLYLQMNSLRAEDTAVYYCARHGYDGTWFAYWGQGTLVTVSSGGGGSGGGGSGGGGSDVLTQSPSSLSASVGDRVTITCRASENIYSYLAWYQQKPGKSPKLLVSNAKTLAEGVPSRFSGSGSGTQFSLTISSLQPEDFATYYCQQHYATPYTFGGGTKLEIKRTVGGGGSGGGGSGGGGSQGQDRHMIRMRQLIDCVDQLKNYVNDLVPEFLPAPEDVETNCEWSAFSCFQKAQLKSANTGNNERIINVCIKKLKRNLWGLAGLNSCPSCDSYEKKPPKEFLERFKSLLQKMIHQHLSSRTHGSEDS

SEQ ID NO: 60 - DNA, 1362 bp, synthetic construct
  source 1..1362: mol_type = other DNA
atgacgtggagggctgccgcctccacgtgcgcggcgctcctgattctgctgtgggcgctgacgaccgaaggtgatctgaaagtagagatgatggcaggggggactcagatcacacccctgaatgacaatgtcaccatattctgcaatatcttttattcccaacccctcaacatcacgtctatgggtatcacctggttttggaagagtctgacgtttgacaaagaagtcaaagtctttgaattttttggagatcaccaagaggcattccgacctggagccattgtgtctccatggaggctgaagagtggggacgcctcactgcggctgcctggaatccagctggaggaagcaggagagtaccgatgtgaggtggtggtcacccctctgaaggcacagggaacagtccagcttgaagttgtggcttccccagccagcagattgttgctggatcaagtgggcatgaaagagaatgaagacaaatatatgtgtgagtcaagtgggttctacccagaggctattaatataacatgggagaagcagacccagaagtttccccatcccatagagatttctgaggatgtcatcactggtcccaccatcaagaatatggatggcacatttaatgtcactagctgcttgaagctgaactcctctcaggaagaccctgggactgtctaccagtgtgtggtacggcatgcgtccttgcatacccccttgaggagcaactttaccctgactgctgctcggcacagtctttctgaaactgagaagacagataatttttccattcattggtggcctatttcattcattggtgttggactggttttattaattgttttgattccttggaaaaagatatgtaacaaatcatcttcagcctatactcctctcaagtgcattctgaaacactggaactcctttgacactcagactctgaagaaagagcacctcatattcttttgcactcgggcatggccgtcttaccagctgcaggatggggaggcttggcctcctgagggaagtgttaatattaatactattcaacaactagatgttttctgcagacaggagggcaaatggtccgaggttccttatgtgcaagccttctttgccttgcgagacaacccagatctttgtcagtgttgtagaattgaccctgctctcctaacagttacatcaggcaagtccatagatgataattccacaaagtctgagaaacaaacccctagggaacactcggatgcagttccggatgccccaatccttcctgtctcccctatctgggaacctcctccagccacaacatcaacaactccagttctatcctcccaacccccaactttactgttacccctacag

SEQ ID NO: 61 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 62 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

(19) United States
(12) Patent Application Publication
(10) Pub. No.: US 2025/0043007 A1
(43) Pub. Date: Feb. 6, 2025

(54) ANTI-B7H6 SCFV ANTIBODY, ITS CODING GENES AND THE APPLICATION THEREOF

(71) Applicants: Zhongwei XU, Beijing (CN); ADVANCED BIOTHERAPEUTICS, LTD, Suzhou (CN)

(72) Inventors: Zhongwei XU, Beijing (CN); Haiyan ZHANG, Beijing (CN)

(21) Appl. No.: 18/778,908

(22) Filed: Jul. 19, 2024

Related U.S. Application Data
(63) Continuation of application No. PCT/CN2023/081789, filed on Mar. 16, 2023.

(30) Foreign Application Priority Data
Apr. 18, 2022 (CN) 202210406162.X

(51) Int. Cl. C07K 16/28; A61K 39/00; C07K 14/54; C07K 14/705; C07K 14/725; C12N 5/0783

(52) U.S. Cl. CPC C07K 16/2827; A61K 39/4611; A61K 39/4631; A61K 39/464411; C07K 14/54; C07K 14/7051; C07K 14/70517; C07K 14/70578; C07K 16/2818; C12N 5/0636; C07K 2317/53; C07K 2317/565; C07K 2317/622; C07K 2317/73; C07K 2317/92; C07K 2319/02; C07K 2319/03; C12N 2510/00

(57) ABSTRACT

The present invention discloses an anti-B7H6 ScFv antibody, its coding genes, and the application thereof, wherein B7H6-CAR-T cells contain antibodies targeting the B7H6 antigen or their antigen binding fragments and contain heavy chain variable regions or light chain variable regions. The heavy chain variable region contains CDR1-3 of the amino acid sequence shown in SEQ ID NO.: 11-13 and/or the light chain variable region contains CDR1-3 of the amino acid sequence shown in SEQ ID NO.: 14-16; or the heavy chain variable region contains CDR1-3 of the amino acid sequence shown in SEQ ID NO.: 17-19 and/or the light chain variable region contains CDR1-3 of the amino acid sequence shown in SEQ ID NO.: 20-22. The antibody and the B7H6-CAR based on this antibody present strong affinity with the B7H6 antigen molecule.

### CROSS REFERENCE TO RELATED APPLICATION

The present application claims the priority of CN 202210406162.X submitted on Apr. 18, 2022, the entire content of which is incorporated herein by reference.

### REFERENCE TO AN ELECTRONIC SEQUENCE LISTING

The contents of the electronic sequence listing (Sequence_listing.xml; Size: 75,166 bytes; and Date of Creation: Apr. 30, 2024) is hereby incorporated by reference in its entirety.

### TECHNICAL FIELD

The present invention relates to the technical field of the biological immunotherapy technology, specifically to Anti-B7H6 ScFv antibody, its coding genes and the application thereof.

### BACKGROUND ART

The B7 family belongs to the immunoglobulin superfamily and is composed of structurally similar cell surface glycoprotein ligands. These ligands are mainly expressed on various immune and non-immune cells and bind to receptors on effector lymphocytes such as T, B, NK, etc., thereby forming “co-stimulatory” or “co-inhibitory” signals to assist in regulating the activation, proliferation, immune response, and other final biological activities of lymphocytes. At present, it is known that 11 members of this family have been discovered, such as B7-2 (interacting with CD28) and B7H1 (i.e. PD-L1, interacting with PD-1). B7H6 is a newly discovered “co-stimulatory” signaling protein in the B7 family, with a size of 51 kDa, the receptor with which it interacts is NKp30 and is mainly expressed on the surface of NK cells. The combination of the both can activate the immune effect of NK cells. Interestingly, B7H6 has been found to be expressed on the surface of many tumor cells, such as lymphoma, leukemia, gastrointestinal tumors, small cell lung cancer, etc., but it is almost not expressed in normal tissue cells; and there is a certain correlation between its expression rate in tumor cells and poor tumor prognosis. Recently, it has also been found that tumor cells can evade immune cell surveillance by downregulating or shedding the B7H6 expression. Therefore, B7H6 is considered a potential ideal target for tumor targeted therapy and prognostic diagnosis.

Chimeric antigen receptor (CAR) T cell therapy has been shown in clinical trials to have long-lasting and significant therapeutic effects on B-cell leukemia. The CAR strategy can target any tumor surface antigen as long as it can generate antigen binding receptors. The preparation of CAR-T and CAR-NK cell therapy targeting B7H6 tumor associated antigens is one of the good choices for the treatment of solid tumors and hematological tumors. There is still an urgent need for B7H6-CAR-T cells and the preparation method thereof to effectively prevent and/or treat cancer or tumors.

The information in the background art is only intended to illustrate the overall background of the present invention and should not be regarded as acknowledging or implying in any form that this information constitutes prior art known to those skilled in the art.

### SUMMARY OF THE INVENTION

To solve the technical problems in the existing technology, the inventor discovered a series of different antibodies targeting B7H6 antigen molecules through in-depth research, wherein CAR-T cells prepared from scFv antibodies Ad02 and Ad05 can efficiently and specifically kill tumor cells expressing B7H6 and transformed cells. In addition, the present invention applies suicide gene structures to CAR-T structures, which can eliminate CAR-T cells when they are not needed, thereby ensuring the safety of its application. Specifically, the present invention includes the following content.

The first aspect of the present invention provides an antibody or antigen binding fragment thereof comprising a heavy chain variable region or a light chain variable region, wherein,
the heavy chain variable region includes antigen complementary determining regions CDR1, CDR2, and CDR3 of the amino acid sequence shown in SEQ ID NO.: 11-13 respectively; and/or
the light chain variable region comprises antigen complementary determining regions CDR1, CDR2, and CDR3 of the amino acid sequence shown in SEQ ID NO.: 14-16 respectively, or
the heavy chain variable region includes antigen complementary determining regions CDR1, CDR2, and CDR3 of the amino acid sequence shown in SEQ ID NO.: 17-19 respectively; and/or
the light chain variable region includes antigen complementary determining regions CDR1, CDR2, and CDR3 of the amino acid sequence shown in SEQ ID NO.: 20-22 respectively.

According to the antibody or the antigen binding fragment thereof of the present invention, preferably, it is capable of specifically binding to the antigen B7H6.

According to the antibody or the antigen binding fragment thereof of the present invention, preferably, the antibody has any one of the amino acid sequences shown in (I), (II), or (III):
a) the amino acid sequences obtained from the heavy chain variable region coding sequence shown in SEQ ID NO.: 23 and/or the light chain variable region coding sequence shown in SEQ ID NO.: 24; or the amino acid sequences obtained from the heavy chain variable region coding sequence shown in SEQ ID NO.: 25 and/or the light chain variable region coding sequence shown in SEQ ID NO.: 26;
b) the amino acid sequence has at least 90%, preferably at least 95%, further preferably at least 98%, and most preferably at least 99% homology with the amino acid sequence obtained from any of the encoding sequences shown in SEQ ID NO.: 23-26;
c) the amino acid sequence obtained by modification, substitution, deletion, or addition of one or more amino acids into the encoding sequence shown in SEQ ID NO.: 23-26;
wherein, the antibody or the antigen binding fragment thereof in (II) or (III) still maintains the ability to specifically bind with the B7H6 antigen.

Preferably, according to the antibody or antigen binding fragment of the present invention, the antibody comprises at least one of monoclonal antibodies, chimeric antibodies, humanized antibodies, or bispecific antibodies; the antigen binding fragments include at least one of Fab fragments, Fab′, F(ab′)2 fragments, single chain variable fragments scFv, scFv-Fc fragments, or single chain antibody ScAb.

The second aspect of the present invention provides a chimeric antigen receptor comprising:
i. the antigen binding domain recognizing the B7H6 antigen, wherein the antigen binding domain comprises the antibody or the antigen binding fragment thereof according to the first aspect;
ii. a transmembrane domain; and
iii. an intracellular signal transduction domain;
Preferably, the chimeric antigen receptor further comprises a hinge area;
Preferably, the chimeric antigen receptor further comprises suicide switch molecules;
Preferably, the chimeric antigen receptor further comprises an intracellular costimulatory domain;
preferably, the chimeric antigen receptor further comprises a fusion fragment, wherein the fusion fragment comprises a cytokine and an anti-PD1-scFv or PD1 antigen binding fragment;
Preferably, the cytokines include IL21;
Preferably, the transmembrane domain is selected from at least one of peptides CD28, NKp30, CDS, DAP10, 4-1BB, DAP12, CD3C, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1, ICOS(CD278), 4-1BB(CD137), GITR, CD40, BAFFR, HVEM (LIGHT), SLAMF7, NKp80 (KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4(CD244, 2B4), CD84, CD96, CEACAM1, CRTAM, Ly9(CD229), CD160(BY55), PSGL1, CD100(SEMA4D), SLAMF6 (NTB-A, Ly108), SLAM(SLAMF1, CD150, IPO-3), BLAME(SLAMF8), SELPLG(CD162), LTBR, PAG/Cbp or any combinations thereof;
Preferably, the intracellular signal transduction domain is selected from at least one of CD8, CD3ζ, CD3δ, CD3γ, CD3ε, FcγRI-7, FcγRIII-7, FcεRIβ, FcεRIγ, DAP10, DAP12, CD32, B7H69a, B7H69b, CD28, CD3C, CD4, b2c, CD137(4-1BB), ICOS, CD27, CD28 δ, CD80, NKp30, OX40 or any combination thereof;
Preferably, the intracellular signaling domain comprises a shortened CD3ζ chain which is retained from at least one ITAM motif of the CD3ζ chain, further preferably the first ITAM motif among the three ITAMs in the CD3ζ chain.

A third aspect of the present invention provides a separated nucleic acid molecule encoding the antibody or the antigen binding fragment thereof according to the first aspect of the present invention, or the chimeric antigen receptor according to the second aspect.

The fourth aspect of the present invention provides a carrier comprising the nucleic acid molecule according to the third aspect.

The fifth aspect of the present invention provides a host cell comprising a carrier according to the fourth aspect.

A sixth aspect of the present invention provides a preparation method for the chimeric antigen receptor according to the second aspect and the method includes culturing host cells according to the fifth aspect.

A seventh aspect of the present invention provides an immunologic effector cell that expresses the antibody or the antigen binding fragment thereof according to the first aspect of the present invention, or the chimeric antigen receptor according to the second aspect;

Preferably, the immunologic effector cells are selected from at least one of leukocytes, monocytes, macrophages, dendritic cells, mast cells, neutrophils, basophils, eosinophils, αβ T cells, γδT cells, natural killer (NK) cells, natural killer T (NKT) cells, B cells, natural lymphoid cell (ILC), cytokine induced killer (CIK) cells, cytotoxic T lymphocytes (CTL), lymphokine activated killer (LAK) cells, T lymphocytes, peripheral blood mononuclear cells, and hematopoietic stem cells.

The eighth aspect of the present invention provides the application of reagents in the preparation of compositions, drugs, preparations, or test kits for the prevention and/or treatment of cancer or tumors, wherein the reagents include: antibodies or antigen binding fragments thereof, wherein the reagents comprise the antibody or the antigen binding fragment thereof according to the first aspect of the present invention or the chimeric antigen receptors according to the second aspect or the immunologic effector cells according to the seventh aspect;

Preferably, the cancer or tumor refers to the cancer or tumor related to the B7H6 expression. More preferably, the cancer or tumor includes myeloid leukemia, acute non lymphocytic leukemia, acute lymphoblastic leukemia, B-cell lymphoma, breast cancer, cervical cancer, transparent cell renal cell carcinoma, protuberant skin fibrosarcoma, gastric sarcoma, gastrointestinal stromal tumor, glioblastoma, leiomyosarcoma, invasive ductal breast cancer, malignant fibrous histiocytoma, melanoma, ovarian serous surface papillary carcinoma, pancreatic cancer, prostate cancer, T-cell acute lymphocytic leukemia, small cell lung cancer or T-cell lymphoma.

The ninth aspect of the present invention provides the application of the antibody or the antigen binding fragments thereof according to the first aspect of the present invention, or the chimeric antigen receptor according to the second aspect or the immunologic effector cells in combination with other drugs according to the seventh aspect. Other medications include but are not limited to diagnostic agents, prophylactic agents, and/or therapeutic agents.

A tenth aspect of the present invention provides a method for preventing and/or treating cancer or tumors and the method includes the step of administering a therapeutically effective amount of a drug combination to a subject in need, wherein the drug combination comprises the antibody or antigen binding fragment thereof or the nucleic acid molecule of the drug combination.

The excellent technical effects of the present invention include but are not limited to:
1. The antibody or the antigen binding fragment thereof of the present invention can specifically bind to human B7H6 antigen molecules (such as binding to the extracellular domain of the antigen molecule, preferably binding to the amino acid residue 25-262 in the extracellular domain of the antigen molecule), demonstrating strong affinity with B7H6 antigen molecules.
2. The CAR structure constructed by the present invention can relieve the inhibitory effect of the tumor microenvironment on specific T cells.
3. The CAR structure constructed by the present invention can promote the formation and proliferation of memory T cells and improve the effectiveness of tumor treatment.
4. The B7H6-CAR-T cells of the present invention have a significant and specific killing effect on B7H6 positive target cells, providing beneficial CAR-T cells for clinical application of the cell therapy.
5. The present invention applies a suicidal gene structure on the CAR-T structure, which can eliminate CAR-T cells when they are not needed, thereby ensuring the safety of its application.
6. The antibody or the antigen binding fragment thereof of the present invention can be applied to the treatment and diagnosis of diseases such as ADCC, ADC and double/multi specific antibodies, which greatly expands the application scope of immunotherapy.

In addition, the present invention also includes the following effects: effectively killing cells expressing B7H6, reducing the growth of cells expressing B7H6, or effectively inducing an immune response against cells expressing B7H6.

### BRIEF DESCRIPTION OF DRAWINGS

FIG. 1 shows the plasmid map of the third-generation lentivirus vector pCDH-EF1 (X6)-MCS-T2A-Puro.

FIG. 2 shows the molecular structure diagram of B7H6-CAR (CD3ζ1).

FIG. 3 shows the molecular structure diagram of B7H6-CAR (CD3ζ).

FIG. 4 shows the molecular structure diagram of B7H6-CAR (CD3) ζ)-aPD1-IL21.

FIG. 5-10 shows the coculture killing curve of the B7H6-CAR-T cell and the target cell U87-B7H6/U87. The effector-target ratio corresponding to the curves in the figure is 0:1 (target only, blank control of target cells), 1:1, 2:1, and 4:1 respectively.

FIG. 11 shows the killing efficiency of B7H6-CAR-T on the target cell U87-B7H6/U87. Each column in the figure corresponds to a effector-target ratio of 1:1, 2:1, and 4:1 from left to right.

### SPECIFIC EMBODIMENTS

Now multiple exampled embodiments in the present invention are described in details, and the detailed description should not be deemed as limitations to the present invention but should be interpreted to be more detailed description on some aspects, features and embodiments of the present invention. Where the embodiments does not indicate specific techniques or conditions, they are performed by techniques or conditions described by literatures in the art (such as Molecule Cloning Experiment Guidelines by J. Sambrook, translated by Huang Peitang et al, third edition, Science press) or by the product specification. Where the used agents or instruments are not indicated with a manufacturer, they can be commercially available normal products.

It should be understood that the term in the present invention is merely used to describe a particular embodiment and is not intended to limit the present invention. In addition, for the numerical range in the present invention, it should be understood as specifically disclosing the upper and lower limits of the range as well as each intermediate value between them. Any stated values or the intermediate values in the state range and each smaller range between any stated value or intermediate value within the stated range is also included in the present invention. The upper and lower limits of these smaller ranges can be independently included or excluded within the range.

Unless otherwise specified, all technical and scientific terms used in the present invention have the same meaning as those commonly understood by conventional technicians in the field of the present invention. Although the present invention merely describes preferred methods and materials, any methods and materials similar or equivalent to those described in present invention can also be used in the implementation or verification of the present invention. All literatures mentioned in the description are incorporated by reference to publicize and disclose methods and/or materials related to the literature. In case of conflict with any incorporated literatures, the content of this description shall prevail.

In the present invention, B7H6, B7 homolog 6 and B7-H6 are interchangeable and refer to ligands of NK cell activation receptor NKp30.

The terms “specific to”, “target” and “specific binding” used herein can be interchangeably used to refer to nonrandom binding reactions between two molecules, such as the antibody binding to the antigen epitope.

The heavy chain variable region and the light chain variable region in the antibodies typically include three complementary determining regions (CDR) and four skeletal regions (FR). Complementary determining regions are connected by skeleton regions. When recognizing antibodies, FR molecules are coiled to bring CDR molecules closer to each other. The complementary determining region is the location where the antibody or antigen binding fragment binds with the antigen, which means that the sequence of the complementary determining region determines the specificity of the antibody. As understood in this field, antibodies are glycoproteins or antigen-binding portions thereof that contain at least two heavy (H) chains and two light (L) chains interconnected by disulfide bonds. The heavy chain includes a variable heavy chain region (VH) and a constant heavy chain region (CH). The light chain includes a variable region (VL) and a constant region (CL). The variable regions of heavy and light chains include the frame region (FR) and the complementary decision region (CDR). The four FRs are relatively conservative, while the CDR regions (CDR1, CDR2, and CDR3) contain highly variable regions.

The “antigen binding fragment” herein refers to a peptide fragment that contains a portion of the intact antibody, such as the antigen binding region or variable region of the intact antibody, and has the characteristic of specifically targeting B7H6. Preferably, it contains at least one CDR in the antibody heavy chain variable region and/or light chain variable region; preferably, it may contain CDR1-3 in a heavy chain variable region and/or CDR1-3 in a light chain variable region. The antigen binding fragments can be prepared through various techniques, including but not limited to hydrolysis and digestion of intact antibody proteins, or expression by host cells containing antigen binding fragments.

The present invention provides the antibody targeting B7H6 or antigen binding fragments, which have good safety and targeting properties and can specifically bind to the extracellular domain of human B7H6. A vector containing the encoding sequence of the antibody or the antigen binding fragment thereof is used to infect immune cells, which can obtain immunologic effector cells with significant killing ability against tumor cells expressing B7H6. These immunologic effector cells can be applied to treat or improve diseases related to B7H6 expression, laying the foundation for the treatment of B7H6 positive tumors.

Without limitations or theoretical constraints, the sequences of the heavy chain variable regions CDR1, CDR2, CDR3, and the light chain variable regions CDR1, CDR2, and CDR3 of antibodies or their antigen binding fragments can be randomly selected within the following range: the heavy chain variable region of the antigen complementary determining regions CDR1, CDR2, and CDR3 of the amino acid sequence shown in SEQ ID NO.:11-13 respectively, and/or the light chain variable region of the antigen complementary determining regions CDR1, CDR2, and CDR3 of the amino acid sequence shown in SEQ ID NO.: 14-16 respectively; or

the heavy chain variable region of the antigen complementary determining regions CDR1, CDR2, and CDR3 in the amino acid sequence shown in SEQ ID NO.: 17-19 respectively; and/or the light chain variable region of the antigen complementary determining regions CDR1, CDR2, and CDR3 in the amino acid sequences as shown in SEQ ID NO.: 20-22 respectively.

In the present invention, the antibody or the antigen binding fragment thereof has any one of the amino acid sequences shown in (I), (II), or (III): (I) the amino acid sequence obtained from the heavy chain variable region coding sequence shown in SEQ ID NO.: 23 and/or the amino acid sequence obtained from the light chain variable region coding sequence shown in SEQ ID NO.: 24; or the amino acid sequence obtained from the heavy chain variable region coding sequence shown in SEQ ID NO.: 25 and/or the light chain variable region coding sequence shown in SEQ ID NO.: 26; (II) the amino acid sequence having at least 90%, preferably at least 95%, further preferably at least 98%, and most preferably at least 99% homology with the amino acid sequence obtained from any of the encoding sequences shown in SEQ ID NO.: 23-26; (III) the amino acid sequence obtained by modification, substitution, deletion, or addition of one or more amino acids into the encoding sequence shown in SEQ ID NO.: 23-26. It should be noted that the homology (sometimes referred to as “identity” herein) sequence mentioned above does not alter the antigen antibody binding characteristics, namely the antibody or the antigen binding fragment thereof selected from the above amino acid sequence still retains the binding activity of the antibody with the antigen B7H6 on the tumor surface. Preferably, changes to the amino acid in (II) and (III) all occur in the frame region (FR region), namely the amino acid sequences containing their respective heavy and light chains CDR1-3 have at least one mutation in at least one frame region of the amino acid sequences obtained from any of the coding sequences shown in SEQ ID NO.: 23-26.

Preferably, the above-mentioned amino acid sequence in the present invention is obtained by subjecting to the coding sequence of the mouse derived antibodies to the host codon preference modification and then expression. In the present invention, subjecting to preference modification refers to the substitution of base sequences with bases based on degenerate codons to meet the needs of different host expressions. The codon preference modification generally does not change the sequence of product proteins or peptides. In the coding sequence of the mouse derived antibody (Ad02), the coding sequence of the heavy chain variable region is shown as SEQ ID NO.: 3, the coding sequence of the light chain variable region is shown as SEQ ID NO.: 5, the amino acid sequence of the heavy chain variable region is shown as SEQ ID NO.: 4, and the amino acid sequence of the light chain variable region is shown as SEQ ID NO.: 6. The present invention also provides an anti-B7H6 monoclonal antibody Ad05. The nucleotide sequence encoding the heavy chain variable region VH is shown in SEQ ID NO.: 7, the amino acid sequence of the heavy chain variable region VH is shown in SEQ ID NO.: 8, the nucleotide sequence encoding the light chain variable region VL is shown in SEQ ID NO.: 9, and the amino acid sequence of the light chain variable region VL is shown in SEQ ID NO.: 10.

Preferably, the antibody comprises at least one of monoclonal antibodies, humanized antibodies, chimeric antibodies, and bispecific antibodies; the antigen binding fragments are at least one of Fab, F (ab′), F (ab′)₂, Fd, single chain antibody scFv, disulfide linked Fv (sdFv), or single domain antibody. Preferably, the antibody or the antigen binding fragment thereof is humanized.

Preferably, the antibody further comprises an antibody constant region; preferably, the antibody constant region is selected from the constant region of any one of IgG1, IgG2, IgG3, IgG4, IgA, IgM, IgE, and IgD.

Preferably, the heavy chain constant region of the antibody constant region is selected from the heavy chain constant region of any one of IgG1, IgG2, IgG3, or IgG4, preferably the heavy chain constant region of IgG4; the light chain constant region of the antibody constant region is κ or λ.

The antibody of the present invention may include an Fc region which is derived from IgG, such as IgG1, IgG2, IgG3, or IgG4.

The term “monoclonal antibody” used herein sometimes is also known as “monoclonal antibody” or mAb, which refers to an immunoglobulin obtained from a pure cell line, has the same structure and chemical properties and is specific to a single antigenic determinant. Monoclonal antibodies are different from conventional polyclonal antibody preparations (usually with different antibodies targeting different determinant clusters), as each monoclonal antibody targets a single determinant cluster on an antigen. In addition to their specificity, the advantage of monoclonal antibodies is that they are obtained through hybridoma or recombinant engineering cell culture without mixing with other immunoglobulins. The modifier “monoclonal” indicates the characteristics of an antibody obtained from a homogeneous antibody population, but this should not be interpreted as requiring any special or specific method to produce the antibody.

Variant antibodies are also included within the scope of the present invention. The present invention does not specifically limit the sequence of variants, as long as they have binding characteristics targeting B7H6 antigen or have antibodies with increased affinity. Other variants with such sequences can be obtained using methods known in the art and are all within the scope of the present invention. A person skilled in the art can modify the amino acid sequence of peptides by utilizing recombination methods and/or synthetic chemistry techniques for producing variant peptides. For example, amino acid substitution can be used to obtain antibodies with further enhanced affinity. Optionally, codon optimization of nucleotide sequences can be used to improve translation efficiency in the expression system used for antibody production. Such variant antibody sequences have sequence identity of 80% or higher (i.e. 85%, 90%, 95%, 96%, 97%, 98%, 99% or greater) with the sequences listed in the present invention. The sequence identity is calculated in relative to the sequences listed in the present invention. Or the best comparison is performed such as through program GAP or using BESTFIT with default gap weights.

The term “modification” used herein refers to that the amino acid modification does not significantly affect or alter the binding characteristics of antibodies containing that amino acid sequence. This type of modification includes substitution, addition, and deletion of amino acids. Preferably, different residue positions vary due to the substitution of conserved amino acids. The antibodies of the present invention may include glycosylation, acetylation, phosphorylation, amidation, derivatization through known protective/blocking groups, protein hydrolysis cleavage, or non-naturally occurring amino acid modifications.

Conservative amino acid substitution refers to the interchangeability of residues with side chains. For example, the amino acid groups with aliphatic side chains are glycine, alanine, valine, leucine, and isoleucine; the amino acid groups with aliphatic hydroxyl side chains are serine and threonine; The amino acid groups with amide side chains are asparagine and glutamine; the amino acid groups with aromatic side chains are phenylalanine, tyrosine, and tryptophan; the amino acid groups with alkaline side chains are lysine, arginine, and histidine; the amino acid groups with sulfur-containing side chains are cysteine and methionine. The preferred conservative amino acid substitution groups are valine-leucine-isoleucine, phenylalanine-tyrosine, lysine-arginine, alanine-valine, glutamic acid-aspartic acid, and asparagine-glutamine. Therefore, one or more amino acid residues in the CDR region of the antibody of the present invention or one or more amino acid residues in the frame region outside the CDR region can be substituted with other amino acid residues from the same side chain family.

Another possible type of variable region modification is the mutation of amino acid residues in the CDR1, CDR2, and/or CDR3 regions of VH and/or VL to improve one or more binding properties (such as affinity) of the target antibody. Mutations can be introduced through targeted mutagenesis or PCR mediated mutagenesis. Preferably the above conservative modifications are introduced. Mutations can be substitutions, additions, or deletions of amino acids, but substitution is preferred. In addition, the variation of residues in the CDR region or the framework region outside the CDR region usually does not exceed one, two, three, four, or five.

The present invention also provides an anti-human B7H6 chimeric antigen receptor CAR, wherein the CAR comprises an antigen binding domain (sometimes referred to as an “antigen recognition region” herein), a hinge area, a transmembrane domain (sometimes referred to as a “transmembrane region” herein) and an intracellular signal transduction domain (sometimes referred to as an “intracellular region” herein) that can recognize B7H6 antigen, wherein the antigen recognition region comprises the antibody or the antigen binding fragment thereof specifically bound to B7H6 as described in the present invention.

In the absence of limitations, the antigen recognition region can be monovalent or multivalent (such as divalent or trivalent). The antigen binding region can be single specific or multi specific (such as bispecific). Bispecificity can be targeting B7H6 and another antigen, or it can be targeting two different epitopes of B7H6. Preferably, the antigen recognition region is a single chain antibody (monovalent or multivalent). The single chain antibody scFv includes a heavy chain variable region and a light chain variable region. The heavy chain variable region and the light chain variable region are connected by linkers to form antibodies. Preferably, the connection method for scFv heavy and light chains is VH-Linker-VL or VL-Linker-VH. In some embodiments, the sequence of Linker can application existing linker sequences. More preferably, the sequence of Linker is the nucleotide sequence shown in SEQ ID NO.: 27.

Preferably, the CAR further includes a signal peptide sequence. Generally speaking, signal peptides are peptide sequences that enable peptides to target the desired site in cells. In some embodiments, signal peptides target the secretion pathways of cells and will allow peptide to be integrated and anchored to the lipid bilayer. In some embodiments, the signal peptide is a membrane localization signal peptide. Preferably, the signal peptide sequence is derived from the signal peptide sequence of CD8a; More preferably, the CD8a signal peptide sequence has an amino acid sequence as shown in SEQ ID NO.: 38.

The “hinge area”, “transmembrane region” and “intracellular region” herein can all be selected from the sequences of hinge areas, transmembrane regions and intracellular regions in existing known CAR-T techniques.

The hinge area of the chimeric antigen receptor is located between the extracellular antigen binding region and the transmembrane region. The hinge area is an amino acid segment that typically exists between two domains of a protein and allows for protein flexibility and relative movement between the two domains. The hinge area can be the hinge area of a naturally occurring protein or part thereof. The hinge area of antibodies (such as IgG, IgA, IgM, IgE, or IgD antibodies) can also be used for chimeric antigen receptors as described herein. Non-naturally occurring peptides can also be used as the hinge area of the chimeric antigen receptor described herein. In some embodiments, the hinge area is a peptide linker. Preferably, the hinge area originates from CD8α. Further preferably, the hinge area of the CD8α has an amino acid sequence as shown in SEQ ID NO.: 40.

The transmembrane region of chimeric antibody receptors can form α Spiral, a complex of more than one α Spirals, αβ Barrel or any other stable structure that can cross domain cell phospholipid bilayers. The transmembrane region can be of natural or synthetic origin. The transmembrane region can originate from CD3ε, CD4, CD5, CD8α, CD9, CD16, CD22, CD28, CD33, CD37, CD45, CD64, CD80, CD86, CD134, CD137, CD154 and α,β or ζ Chain of T cell receptors. Preferably, the transmembrane region originates from CD8α. Preferably, the transmembrane region of the CD8α has an amino acid sequence as shown in SEQ ID NO.: 42.

Preferably, the intracellular region of the chimeric antigen receptor includes a signal transduction region and/or a costimulatory signal transduction region. The number of signal transduction areas and/or costimulatory signal transduction areas can both be one or more.

The intracellular signaling pathway is responsible for the activation of at least one normal effector function in immunologic effector cells that express chimeric antigen receptors. For example, the effector function of T cells can be cell lysis activity or auxiliary activity, including the cytokine secretion. Although the entire intracellular signal transduction region can usually be utilized, in many cases, the application of the entire chain is unnecessary. As for using the truncated portion of the intracellular signal transduction region, this truncated portion can be used instead of the complete chain as long as its transduction effector functional signal is used. Therefore, the intracellular signal transduction region includes any truncated form of the intracellular signal transduction region that is sufficient to transduce effector functional signals. In some embodiments, the signal transduction area originates from at least one of CD3ζ, FcRγ (FCER1G), FcRβ(Fc)εRib), CD3γ, CD3δ, CD3ε, CD5, CD22, CD137, B7H69a, B7H69b and CD66d. Preferably, the intracellular region originates from human CD3ζ intracellular region. In some embodiments, the intracellular signal transduction domain comprises a shortened CD3ζ chain, abbreviated as CD3ζ1. The CD3ζ1 only retained the first ITAM motif in the three ITAMs (Immunoacceptor Tyrosine based Activation Motifs) of the CD3ζ chain and the CD3ζ1 has the amino acid sequence shown in SEQ ID NO.: 46. In another embodiment, the human CD3ζ intracellular region herein has an amino acid sequence as shown in SEQ ID NO.: 48. Unlike the prior art, the second and third motifs of the three ITAMs in the CD3ζ chain herein is not subject to base mutation but rather the direct deletion of the second and third motifs and the retained is only the first ITAM motif in the CD3ζ chain, thereby obtaining stronger and more persistent tumor suppressive activity.

In addition to the stimulation of antigen-specific signals, many immunologic effector cells also require costimulation to promote cell proliferation, differentiation and survival as well as the effector function of activated cells. The “costimulatory signal transduction region” can be the cytoplasmic portion of costimulatory molecules. The term “costimulatory molecule” refers to an associated binding partner on immune cells (such as T cells) that specifically binds to costimulatory ligands, thereby mediating the costimulatory responses by immune cells such as but not limited to proliferation and survival. The costimulatory signal transduction area can originate from at least one intracellular signaling regions of CARD11, CD2, B7H6, CD27, CD28, CD30, CD40, CD54, CD83, OX40, CD137, CD134, CD150, CD152, CD223, CD270, PD-L2, PD-L1, CD278, DAP10, LAT, NKD2C, SLP76, TRIM, FcεRIγ, MyD88 and 4-1BB. In some embodiments, the costimulatory signal transduction area originates from 4-1BB. In some embodiments, the 4-1BB costimulatory signal transduction region contains the amino acid sequence shown in SEQ ID NO.: 44.

Preferably, the nucleotide acid sequences and amino acid sequences of the CAR are selected from at least one or a combination of the following sequences:
1. The amino acid sequence is shown in SEQ ID NO.: 28, and its coding sequence is shown in SEQ ID NO.: 29;
2. The amino acid sequence is shown as SEQ ID NO.: 30, and its coding sequence is shown as SEQ ID NO.: 31;
3. The amino acid sequence is shown in SEQ ID NO.: 32, and its coding sequence is shown in SEQ ID NO.: 33;
4. The amino acid sequence is shown in SEQ ID NO.: 34, and its coding sequence is shown in SEQ ID NO.: 35.

In order to address the various toxic side effects associated with CAR-T cell therapy and increase the safety of CAR-T cell therapy, the chimeric antigen receptor CAR designed by the inventor further includes the “suicide switch” RQR8 molecule which has an amino acid sequence as shown in SEQ ID NO.: 52 and the coding sequence as shown in SEQ ID NO.: 51. The RQR8 molecule is fused with the intracellular signaling domain CD3ζ in the B7H6-CAR structure through a T2A linker peptide with auto cleavage function. The T2A linker peptide sequence is not particularly limited. In the specific embodiment, it has an amino acid sequence as shown in SEQ ID NO.: 50, and its coding sequence is as shown in SEQ ID NO.: 49.

Preferably, the RQR8 molecule carries two CD20 antigenic epitopes and target to CD20 using anti CD20 rituximab to activate antibody dependent cell-mediated cytotoxicity (ADCC) and complement mediated cytotoxicity (CDC), inducing T cell apoptosis. When necessary, the application of drugs such as rituximab can achieve the elimination of CAR-T cells, thereby increasing the safety of CAR-T cell therapy.

It should be noted that the CAR structure of the present invention introduces a fusion fragment which includes cytokines and anti-PD1 antibodies or their antigen binding fragments. Preferably, the anti-PD1 antibody or the antigen binding fragment thereof has an amino acid sequence as shown in SEQ ID NO.: 56, and its coding sequence is as shown in SEQ ID NO.: 55. Preferably, the cytokine is an interleukin that regulates the response of CD8+T cells, further preferably the cytokine is IL-21, which has an amino acid sequence as shown in SEQ ID NO.: 59 and a coding sequence as shown in SEQ ID NO.: 58. The inventor found through research that the above CAR structure can relieve the inhibitory effect of the tumor microenvironment on specific T cells. While exerting the function of PD1 antibody, it promotes IL-21 to target tumor specific T cells and improves the formation and proliferation of memory T cells, thus improving the effectiveness of tumor treatments.

The present invention provides a separated nucleic acid that encodes an antibody or antigen binding fragment thereof, or a chimeric antigen receptor as previously described.

The present invention provides a carrier comprising the isolated nucleic acid as described in the present invention. The vector can be an expression vector or a cloning vector. In some embodiments, the vector is a viral vector. Virus vectors include but are not limited to adenovirus vectors, adeno-associated virus vectors, lentivirus vectors, retrovirus vectors, cowpox vectors, herpes simplex virus vectors, and their derivatives.

The present invention provides a host cell comprising the aforementioned carrier. The suitable host cells for cloning or expressing DNA are prokaryotic cells, yeast cells, or higher eukaryotic cells. Common examples of prokaryotic host cells include Escherichia coli, Bacillus subtilis, etc. The commonly used eukaryotic host cells include yeast cells, insect cells, mammalian cells, etc.

The present invention provides a method for preparing an anti-human B7H6 chimeric antigen receptor CAR, comprising culturing the above host cells. Preferably, the culture conditions of the preparation method are sufficient to enable host cells to express the anti-human B7H6 chimeric antigen receptor CAR.

The present invention provides an immunologic effector cell that expresses a specific antibody of B7H6 or the antigen binding fragment thereof, or an anti-human B7H6 chimeric antigen receptor CAR.

In the present invention, “immunologic effector cells” are immune cells that can perform immune effector functions. In some embodiments, immunologic effector cells express at least FcγRIII and execute ADCC effector functions. Examples of immunologic effector cells that mediate ADCC include peripheral blood mononuclear cells (PBMC), natural killer (NK) cells, monocytes, cytotoxic T cells, neutrophils, and eosinophils. Preferably, the immunologic effector cells are selected from at least one of immune cells cultured and differentiated from pluripotent stem cells or embryonic stem cells, T lymphocytes, NK cells, peripheral blood mononuclear cells (PBMC) and hematopoietic stem cells. More preferably, the immunologic effector cells are T lymphocytes (similar to T cells). In some embodiments, T cells can be CD4+/CD8−, CD4−/CD8+, CD4+/CD8+, CD4−/CD8−, or a combination of them. In some embodiments, T cells produce IL-2, IFN, and/or TNF when expressing chimeric antigen receptors and binding to target cells. In some embodiments, CD8+T cells lyse antigen-specific target cells when expressing chimeric antigen receptors and binding to target cells.

The present invention provides a method for preparing immunologic effector cells, which comprises infecting immunologic effector cells with the isolated nucleic acids or carriers as described in the present invention. Preferably, the present invention prepares genetically modified immunologic effector cells by introducing chimeric antigen receptors into immunologic effector cells (such as T cells).

It should be noted that the method of introducing nucleic acids or vectors into mammalian cells is known in this field, and the vectors can be transferred into immunologic effector cells through physical, chemical, or biological methods. The physical methods used to introduce carriers into immunologic effector cells include calcium phosphate precipitation, liposome transfection, particle bombardment, microinjection, electroporation etc. Chemical means for introducing nucleic acids or carriers into immunologic effector cells include colloidal dispersion systems, such as macromolecular complexes, nanocapsules, microspheres, beads, and lipid based systems (including oil in water lotion, micelles, mixed micelles, and liposomes). An exemplary colloidal system used as an in vitro delivery medium is liposomes (such as artificial membrane vesicles). Biological methods for introducing nucleic acids or vectors into immunologic effector cells include the application of DNA and RNA vectors. Virus vectors have become the most widely used method for inserting genes into mammalian cells, such as human cells. In some embodiments, transfected or transfected immunologic effector cells proliferate in vitro after introducing nucleic acids or vectors.

In some embodiments, the preparation also includes further evaluation or screening of transduced or transfected immunologic effector cells to select modified immunologic effector cells.

The present invention further provides a drug or drug combination comprising at least one of an antibody specifically binding to B7H6 or antigen binding fragment thereof, a nucleic acid, a carrier, a chimeric antigen receptor CAR, an anti-human B7H6 chimeric antigen receptor CAR obtained by a preparation method of the chimeric antigen receptor CAR, immunologic effector cell, and an immunologic effector cell obtained by a preparation method of the immunologic effector cell.

In some embodiments, the drug composition further includes pharmaceutically acceptable carriers.

Drug compositions can be prepared in the form of freeze-dried formulations or aqueous solutions by mixing active agents with optional pharmaceutically acceptable carriers of desired purity. Pharmaceutically acceptable carriers are non-toxic to the recipient at the used dosage and concentration and may include at least one of buffering agents, antioxidants, preservatives, isotopes, stabilizers and surfactants. In addition, in order for drug compositions to be suitable for in vivo administration, they must be sterile. The drug composition can be made sterile by filtering through sterile filtration membranes.

In some embodiments, the drug composition may contain at least one additive of cytotoxic agents, chemotherapeutic agents, cytokines, immunosuppressants, growth inhibitors, and active agents required for specific indications to be treated. The specific amount of additives can be adjusted according to actual needs.

The present invention also provides the application of reagents in the preparation of drugs or drug compositions for treating or improving cancer, wherein the reagents are selected from at least one of the antibody specifically binding to B7H6 or antigen binding fragment thereof, the nucleic acid, the carrier, the host cell, the anti-human B7H6 chimeric antigen receptor CAR, the anti-human B7H6 chimeric antigen receptor CAR obtained by the preparation method of the anti-human B7H6 chimeric antigen receptor CAR, the immunologic effector cells, and the immunologic effector cells obtained by the preparation method of the immunologic effector cells.

Preferably, the treatment or improvement of cancer refers to the ability to stimulate or enhance the immune function of cancer patients.

Preferably, the cancer refers to a cancer associated with B7H6 expression.

Herein, “cancer related to B7H6 expression” refers to diseases directly or indirectly caused by abnormal B7H6 expression, usually referring to diseases caused by B7H6 overexpression. Preferably, the cancer or tumor includes but is not limited to myeloid leukemia, acute non lymphocytic leukemia, acute lymphoblastic leukemia, B-cell lymphoma, breast cancer, cervical cancer, clear cell renal cell carcinoma, dermatofibrosarcoma protuberans, gastric sarcoma, gastrointestinal stromal tumor, glioblastoma, leiomyosarcoma, invasive ductal breast cancer, malignant fibrous histiocytoma, melanoma, ovarian serous surface papillary cancer, pancreatic cancer, prostate cancer, T-cell acute lymphocytic leukemia, small cell lung cancer or T-cell lymphoma.

The present invention also provides a method for treating/preventing cancer, comprising the step of administering a therapeutic effective amount of a drug to a subject in need, wherein the drug comprises at least one of an antibody specifically binding to B7H6 or antigen binding fragment, a separated nucleic acid, a carrier, a host cell, an anti-human B7H6 chimeric antigen receptor CAR, an anti-human B7H6 chimeric antigen receptor CAR, an immunologic effector cell and the immunologic effector cells obtained by the preparation method of the immunologic effector cells.

The terms “subject” and “patient” used herein are interchangeably used to refer to any animal that may require antibody related preparations or drugs or treatments described herein. The subjects and patients include but are not limited to, primates (including humans), canines, cats, mice, and other mammalian subjects. Preferably, the subject is a human.

In the present invention, the term “treatment” refers to therapeutic treatment and preventive or prophylactic measures aimed at preventing or slowing down (reducing) unexpected physiological changes or disorders, such as the progression of autoimmune diseases. Beneficial or expected clinical outcomes include, but are not limited to, the following results, whether detectable or undetectable, including relief of symptoms, reduction in disease severity, stability of disease status (i.e. not worsening), delay or slowing of disease progression, improvement or slowing of disease status, and alleviation (whether partial or complete). “Treatment” also refers to the extended survival period compared to the expected survival period when not receiving treatment. Those who need treatment include those who already have symptoms or disorders, as well as those who are prone to symptoms or disorders, or those who need to prevent them.

The term “effective dose” used herein refers to the amount of drug or agent that triggers biological or pharmaceutical responses, such as those pursued by researchers or clinical physicians, in tissues, systems, animals, or humans. In addition, the term “therapeutic efficacy” refers to the amount of improved treatment, cure, prevention, or reduction that causes disease, illness, or side effects, or a decrease in the rate of disease or condition progression, compared to the corresponding subjects who did not receive that amount. This term also includes the amount that effectively enhances normal physiological function within its scope. Usually, the effective dosage herein varies based on various factors, such as the given drug or compound, pharmaceutical formulation, route of administration, type of disease or illness, treated subjects, etc., but can still be routinely determined by those skilled in the art. The effective amount of the compound in the present invention can be easily determined by those skilled in the art through conventional methods known in the art.

The present invention also provides the application of antibodies or their antigen binding fragments, chimeric antigen receptors, or immunologic effector cells in combination with other drugs according to the present invention. Preferably, the other drugs include diagnostic agents, prophylactic agents, and/or therapeutic agents. Furthermore, the other drugs are CD20 targeted antibody drugs, which include but are not limited to: rituximab, atozumab, oxfamumab and teimozumab, etc.

### Embodiment 1

This embodiment is the preparation and sequence analysis of a mouse monoclonal antibody against B7H6 antigen protein.

The present invention uses B7H6 antigen protein to immunize BALB/c mice. After cell fusion, initial screening, and re-screening, a series of hybridoma cell lines capable of producing anti-B7H6 monoclonal antibodies are obtained. The amino acid sequence of the precursor protein of the B7H6 antigen is shown in SEQ ID NO.: 1, where amino acid residues 25-262 are the extracellular domain of the B7H6 antigen, as shown in SEQ ID NO.: 2. The B7H6 antigen protein used for immunizing mice is a recombinant human B7H6 protein (His labeled), with an amino acid sequence being the B7H6 antigen extracellular domain sequences (SEQ ID NO.: 2).

Hybridoma cell lines that produce anti-B7H6 monoclonal antibodies are cultured following by collecting cells and extracting RNA to obtain cDNA sequences encoding anti-B7H6 monoclonal antibodies through RT-PCR. Then, the variable regions of heavy and light chains are cloned using PCR method to connect the PCR products to T-vector. The VH and VL sequences of the variable regions of heavy and light chains of anti-B7H6 monoclonal antibodies are sequenced and further compared and confirmed by using the Uniprot database, thereby selecting two monoclonal antibody clones Ad02 and Ad05 for subsequent experiments.

The nucleotide sequence of VH of anti-B7H6 monoclonal antibody Ad02 is shown in SEQ ID NO.: 3 and its encoded amino acid sequence is shown in SEQ ID NO.: 4. The nucleotide sequence of the VL of Ad02 monoclonal antibody is shown in SEQ ID NO.: 5, and its encoded amino acid sequence is shown in SEQ ID NO.: 6.

The nucleotide sequence of VH of anti-B7H6 monoclonal antibody Ad05 is shown in SEQ ID NO.: 7, and its encoded amino acid sequence is shown in SEQ ID NO.: 8. The nucleotide sequence of the VL of Ad05 monoclonal antibody is shown in SEQ ID NO.: 9, and its encoded amino acid sequence is shown in SEQ ID NO.: 10.

Further analysis is conducted on the amino acid sequences of VH and VL of the two monoclonal antibodies to determine their complementary determining regions (CDR), as shown in Table 1.

TABLE 1
CDR analysis of VH and VL domains
of Anti-B7H6 monoclonal antibodies

| monoclonal | V | CDE | Amino acid | Sequence |
|---|---|---|---|---|
| antibody | region | region | sequence | number |
| Ad02 | VH | CDR1 | SSYIS | 11 |
| | | CDR2 | IYAGTGNT | 12 |
| | | CDR3 | PGEGSPFAY | 13 |
| | VL | CDR1 | QDISNY | 14 |
| | | CDR2 | YTSRLYS | 15 |
| | | CDR3 | QQGDTLPYT | 16 |
| Ad05 | VH | CDR1 | SHYMS | 17 |
| | | CDR2 | INNKDGIT | 18 |
| | | CDR3 | QPSSPYYYAMDY | 19 |
| | VL | CDR1 | KSVSTSGYSY | 20 |
| | | CDR2 | LASNLES | 21 |
| | | CDR3 | QHSRELPPT | 22 |

### Embodiment 2

The synthesis of the coding nucleotide for Anti-B7H6 scFv is as follows.

Firstly, the VH and VL nucleotide sequences coding for monoclonal antibodies Ad02 and Ad05 are used to perform humanized codon optimization for the synthesis of the corresponding nucleotide sequences coding for Anti-B7H6 scFv. The nucleotide sequences coding for VH and VL of monoclonal antibody Ad02 after human source codon optimization are shown in SEQ ID NO.: 23 and SEQ ID NO.: 24, respectively. The nucleotide sequences coding for VH and VL of monoclonal antibody Ad05 after human source codon optimization are shown in SEQ ID NO.: 25 and SEQ ID NO.: 26, respectively. The structures of Anti-B7H6 scFv of the synthesized two monoclonal antibodies are VL-(G4S)4 linker- and VH. The coding nucleotide sequence of the (G4S)4 linker is shown in SEQ ID NO. 27.

### 2. Construction of Lentiviral Expression Plasmid for B7H6-CAR

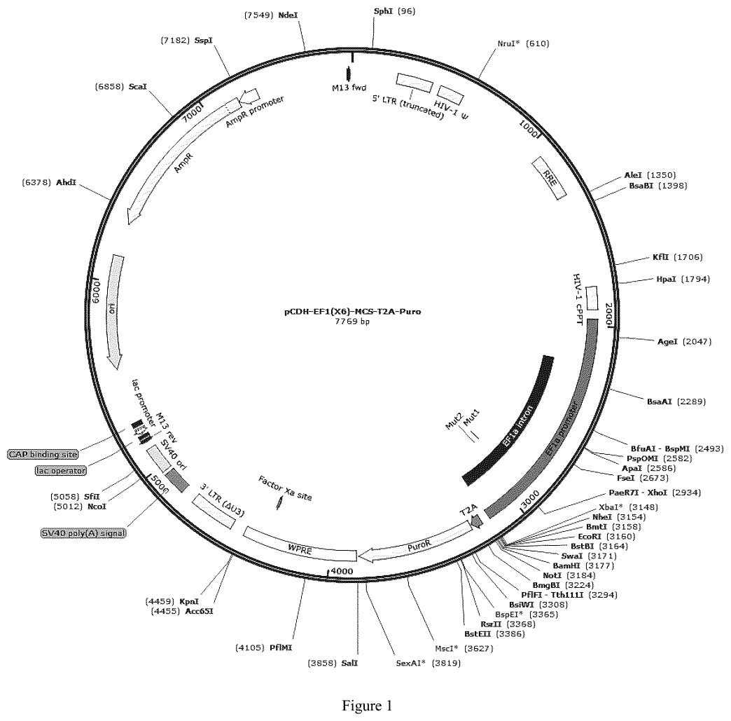

The lentiviral expression plasmid for B7H6-CAR is constructed using conventional techniques. The used vector skeleton is all third-generation lentiviral expression vector pCDH-EF1 (X6)-MCS-T2A-Puro from our company. The map is shown in FIG. 1, and the linearized cleavage sites of the vector are XbaI and SalI. The full length DNA sequence of B7H6-CAR (including N-terminal KOZAC sequence) is inserted between these two cleavage sites.

Four types of B7H6-CAR lentiviral expression plasmids of three different structures are constructed.

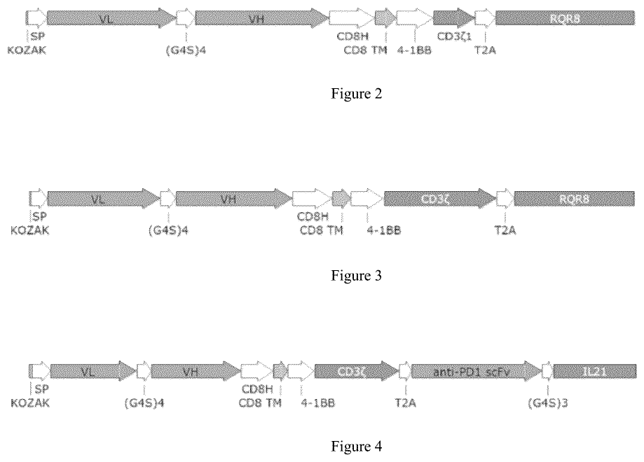

Among them, three types of B7H6-CAR lentiviral expression plasmids are constructed using scFv of the monoclonal antibody Ad02, i.e. (1) B7H6-CAR (CD3ζ1), whose molecular structure is shown in FIG. 2 and full-length amino acid sequence and full-length DNA sequence (including N-terminal KOZAC sequence) are shown in SEQ ID NO.: 28 and SEQ ID NO.: 29, respectively; (2) B7H6-CAR(CD3ζ), whose molecular structure is shown in FIG. 3 and full-length amino acid sequence and full-length DNA sequence (including N-terminal KOZAC sequence) are shown in SEQ ID NO.: 30 and SEQ ID NO.: 31, respectively; (3) B7H6-CAR(CD3ζ)-aPD1-IL21, whose molecular structure of is shown in FIG. 4 and full-length amino acid sequence and full-length DNA sequence (including N-terminal KOZAC sequence) are shown in SEQ ID NO.: 32 and SEQ ID NO.: 33, respectively. In this structure, anti-PD1 scFv is fused with IL21 via a (G4S)3 linker and is connected with CD3ζ structural domain in the previous CAR structure through the auto cleavage linker peptide T2A.

For the monoclonal antibody Ad05, a lentivirus expression plasmid is constructed by using its scFv, whose molecular structure is shown in FIG. 2 as B7H6-CAR (CD3ζ1). The full-length amino acid sequence and full-length DNA sequence (including N-terminal KOZAC sequence) are respectively shown in SEQ ID NO.: 34 and SEQ ID NO.: 35.

So far, the first generation CAR-T only had one intracellular signaling component (CD3)(or FcRγ); the second generation CAR-T is added with the costimulatory domains such as CD28 or 4-1BB; the third-generation CAR-T is simultaneously added with two costimulatory domains, i.e. CD28 and 4-1BB; the fourth generation CAR-T is added with coexpressed cytokines such as IL-2 on the basis of the second generation; the fifth generation CAR-T is also based on the second generation and is added with costimulatory domains that activate other signaling pathways, such as the structural domain where IL2-2Rβ intracellularly binds to SAAT3/5. Generations of diverse structural designs have provided CAR-T cell therapy with infinite vitality and future.

In the present invention, four types of B7H6-CAR in the three constructed structures adopt a second-generation CAR main structure, which contains two intracellular signal domains 4-1BB and CD3ζ (CD3ζ1), where the intracellular signaling component CD3ζ used in the B7H6-CAR(CD3)ζ) structure is a full-length CD3ζ chain and contains three natural ITAM activation motifs (Immunoreceptor Tyrosine based Activation Motifs), which may be more prone to CAR-T cell depletion; only the first ITAM motif in the CD3ζ chain (referred to as CD3ζ1) is retained in the B7H6-CAR(CD3ζ1) structure to obtain stronger and more persistent tumor suppressive activity.

Programmed death protein (PD1) is an immune checkpoint receptor expressed on the surface of T cells. Tumor cells inhibit the killing effect of tumor infiltrating T cells by expressing their ligand PD-L1 (PD-L2) and binding to it. The third CAR structure (B7H6-CAR(CD3ζ)-aPD1-IL21 of the present invention is first introduced with anti-PD1 scFv (anti-PD1 scFv) to relieve the inhibitory effect of the tumor microenvironment on specific T cells. IL-21 is one of the important cytokines that regulate the response of CD8+T cells and can induce the expansion and differentiation of dry memory CD8+T cells. In this structure, IL-21 is further fused with anti-PD1scFv, which not only exerts the function of PD1 antibody but also promotes IL-21 to target tumor specific T cells, promote the formation and proliferation of memory T cells and improve the effectiveness of tumor treatments.

CAR-T cell therapy is typically accompanied with multiple toxic side effects. In order to increase the safety of CAR-T cell therapy, the present invention utilizes the two CAR molecular structures B7H6-CAR(CD3ζ1) and B7H6-CAR(CD3ζ) all incorporate the “suicide switch” RQR8 molecule which connects to the intracellular signaling domain CD3ζ in the B7H6-CAR structure through a T2A linker peptide with auto cleavage function. The RQR8 molecule carries two CD20 antigenic epitope peptides, which are targeted to CD20 using anti CD20 rituximab to activate antibody dependent cell-mediated cytotoxicity (ADCC) and complement mediated cytotoxicity (CDC), inducing T cell apoptosis. When necessary, the application of rituximab can achieve the elimination of CAR-T cells, thereby increasing the safety of CAR-T cell therapy.

The sequence numbers corresponding to the amino acid and nucleotide sequences of each fragment in the four B7H6-CAR molecules are shown in Table 2, where SP is the CD8α signal peptide, CD8H is the CD8α hinge area, CD8TM is the CD8α transmembrane region, 4-1BB and CD3ζ All are intracellular signal transduction domains.

TABLE 2
Corresponding Table of Component Sequences
in the B7H6-CAR Molecular Structure

| | Amino acid | |
|---|---|---|
| | sequence | Nucleotide |
| Fragment name | number | sequnce |
| VH domain of the Ad02 | 4 | 23 |
| monoclonal anti-BTH6 scFv | | |
| VH domain of the Ad02 | 6 | 24 |
| monoclonal anti-BTH6 scFv | | |
| VH domain of the Ad02 | 8 | 25 |
| monoclonal anti-BTH6 scFv | | |
| VH domain of the Ad02 | 10 | 26 |
| monoclonal anti-BTH6 scFv | | |
| KOZAK | / | 36 |
| (G4S)4 Linker | / | 27 |
| SP(CD8a signal peptide) | 38 | 37 |
| CD8H(CD8a) hinge area | 40 | 39 |
| CD8TM(CD8a | 42 | 41 |
| transmembrane domain) | | |
| 4-1bb | 44 | 43 |
| CD3ζ1 | 46 | 45 |
| CD3ζ | 48 | 47 |
| T2A | 50 | 49 |
| RQR8 | 52 | 51 |
| CD20 antigen | 54 | 54 = 3 |
| epitope peptide | | |
| Anti-PD1 scFv | 56 | 55 |
| (G4S)3 Linker | / | 57 |
| IL-21 | 59 | 58 |

### Embodiment 3

The lentivirus packaging adopts the conventional four plasmid system in the art, among which three auxiliary plasmids are pMDLg/pRRE, pRSV-Rev and pMD2. G. the 293T cell are used as lentiviral packaging cells. The plasmid dosage ratio of the lentivirus expression plasmids carrying CD7-Blocker, the 293T cell co-transfected with pMDLg/pRRE, pRSV-Rev and pMD2.G is 7.5:9:9:3.5; the four plasmid dosages are 7.5 ug, 9 ug, 9 ug, and 3.5 ug respectively. The dosage of transfection reagent PEI (ug) is three times the total amount of four plasmids. For T75 culture bottles, the dosage of PEI is 87 ug (1 ug/ul, 87 ul).

After four plasmids cotransfect with the 293T cell, the cell culture medium is collected 48 hours later. After centrifugation (2000 rpm, 15 min), the supernatant is taken and filtered through a 0.45 um filter. The supernatant is concentrated using ultracentrifugation (20000 rpm, 2h), and then the virus precipitate resuspended with the corresponding volume of culture medium according to the dilution ratio, which is divided and stored at −80° C. for cryopreservation.

For the titer determination of B7H6-CAR lentivirus, the lentivirus stock solution or concentrate is subjected to a series of gradient dilutions and transfected into the 293T cell. After 48 hours, the transfection efficiency is detected by flow cytometry to calculate the activity titer of the lentivirus.

### Embodiment 4

This embodiment is the affinity identification between Anti-B7H6 scFv and B7H6 antigen molecules and details are as follows.

The lentivirus of B7H6-CAR (CD3ζ1) in the two monoclonal antibodies Ad02 and Ad05 is transfected into the 293T cell with a series of different MOI values. After 4 days, flow cytometry is used to detect the positive rate of B7H6-CAR in the 293T cell and the ratio of the 293T cell bound to B7H6 antigen proteins. The affinity rate between Anti-B7H6 scFv and B7H6 antigen protein in B7H6-CAR-the 293T cell is calculated to demonstrate the affinity between Anti-B7H6 scFv and B7H6 antigen.

The B7H6 antigen protein is a recombinant human B7H6 protein with His tag in Embodiment 1. During flow cytometry detection, the B7H6 antigen protein is first incubated with B7H6-CAR-the 293T cell, and then fluorescent labeled anti His mouse monoclonal antibody is used to detect the B7H6 antigen protein binding to the 293T cell.

The results are shown in Table 3.

TABLE 3
Affinity detection between Anti-B7H6 scFv and B7H6 antigen molecules

| | Anti-B7H6 scFv source | | | | | | | |
|---|---|---|---|---|---|---|---|---|
| | Ad02 monoclonal antibody | | | | Ad05 monoclonal antibody | | | |
| | MOI values of transducing virus | | | | | | | |
| | 1 | 2 | 5 | 10 | 1 | 2 | 5 | 10 |
| Positive rate of B7H6-CAR in | 35.38 | 52.97 | 70.80 | 78.95 | 1.51 | 2.21 | 4.00 | 7.26 |
| 239 T cells (%) | | | | | | | | |
| Percentage of 239 T cells | 28.69 | 41.82 | 65.25 | 68.97 | 1.41 | 1.80 | 3.03 | 4.09 |
| binding with B7H6 antigen (%) | | | | | | | | |
| Affinity of Anti-B7H6 scFv with | 81.09 | 78.95 | 92.16 | 87.36 | 91.56 | 81.44 | 75.75 | 56.34 |
| the B7H6 antigen (%) | | | | | | | | |
| Affinity average value of Anti-B7H6 | 84.89 | | | | 76.27 | | | |
| scFv with the B7H6 antigen (%) | | | | | | | | |

The results showed that the affinity rates of anti-B7H6 scFv of Ad02 and Ad05 monoclonal antibodies with B7H6 antigen are 84.89% and 76.27%, respectively. In subsequent killing experiments, only the scFv of Ad02 having stronger affinity with the B7H6 antigen protein is used to construct CAR-T.

### Embodiment 5

This embodiment is an in vitro killing test of B7H6-CAR-T cells against B7H6 positive target cells and details are as follows.

In order to further verify the specificity of B7H6-CAR-T cells in killing B7H6 positive target cells. Firstly a B7H6 antigen overexpressing U87-B7H6-eGFP cell line is constructed using U87 cells negative for B7H6 antigen. The killing effect of B7H6-CAR-T cells on B7H6 positive target cells U87-B7H6-eGFP (U87-B7H6 for short) is analyzed using RTCA instrument.

The coding sequence of the B7H6 antigen molecule used to construct the U87-B7H6 eGFP cell line is the DNA coding sequence of the B7H6 antigen precursor protein (SEQ ID NO.: 60) and the amino acid sequence of the eGFP molecule thereof is shown in SEQ ID NO.: 61 with its DNA coding sequence shown in SEQ ID NO.: 62. The B7H6 molecule is connected to the eGFP molecule by an auto cleavage peptide T2A (SEQ ID NO.: 49, SEQ ID NO.: 49, SEQ ID NO.: 50). After adding KOZAK sequence (36) to the N-terminus of B7H6-T2A-eGFP structure, it is inserted between the XbaI and SalI cleavage sites of the lentiviral vector pCDH-EF1(X6)-MCS-T2A-Puro to construct a B7H6 overexpression lentiviral vector. According to conventional methods, B7H6-T2A-eGFP is transduced into U87 cells, and eGFP is used as a screening and detection marker for transduced cells.

The killing test used RTCA (Real Time Cellular Analysis) to detect the killing effect of B7H6-CAR-T cells on B7H6 target cells in real-time.

The CAR structures of three B7H6-CAR-T cells used for killing experiments are respectively B7H6-CAR(CD3ζ1), B7H6-CAR(CD3)ζ) and B7H6-CAR(CD3ζ)-aPD1-IL21 (whose scFv sequences are all derived from monoclonal antibody Ad02), and the corresponding CAR-T cells are respectively referred to as CAR (CD3ζ1)-T, CAR(CD3ζ)-T and CAR (aPD1)-T, whose positive rates of CAR detected by flow cytometry are 51.28%, 45.26%, and 68.98%, respectively.

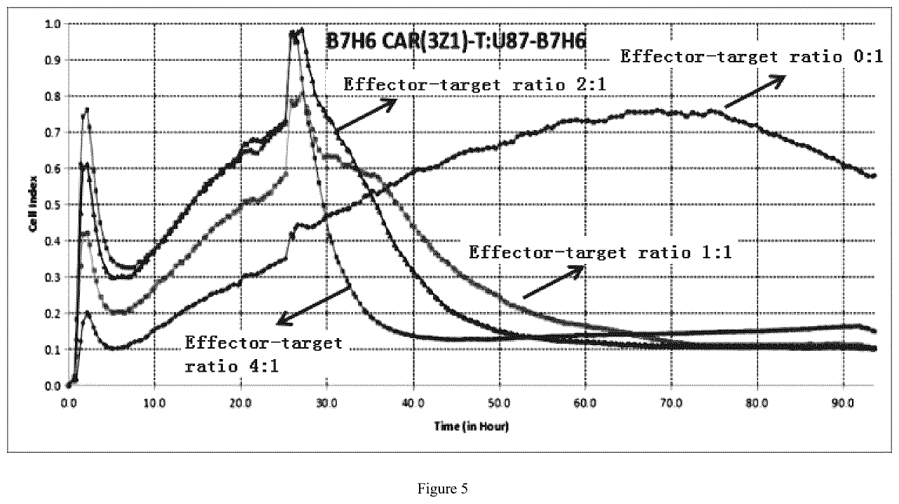

The setup and killing curve of the effect target coculture experimental groups for the killing experiment are shown in Table 4 and FIGS. 5-10.

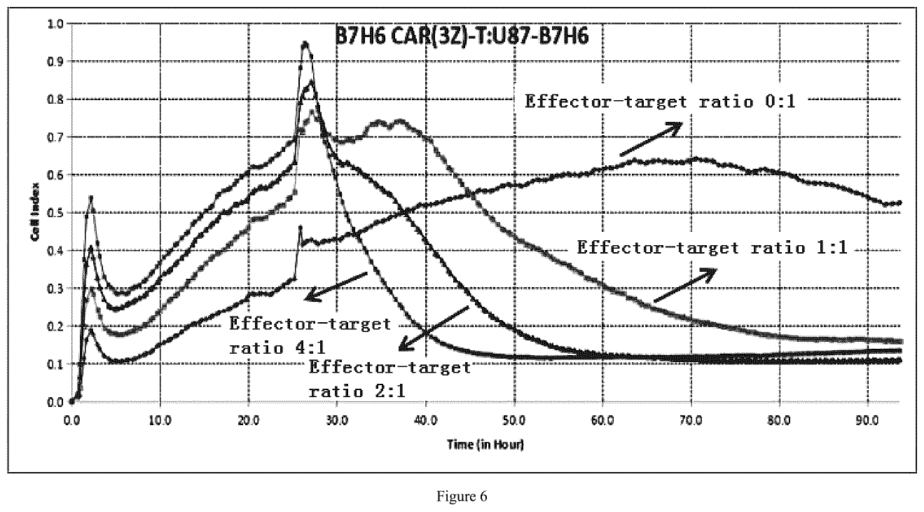

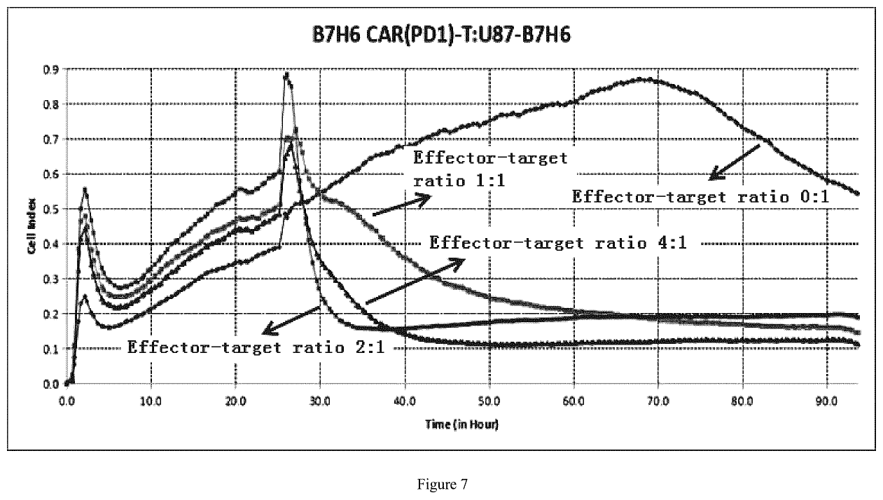

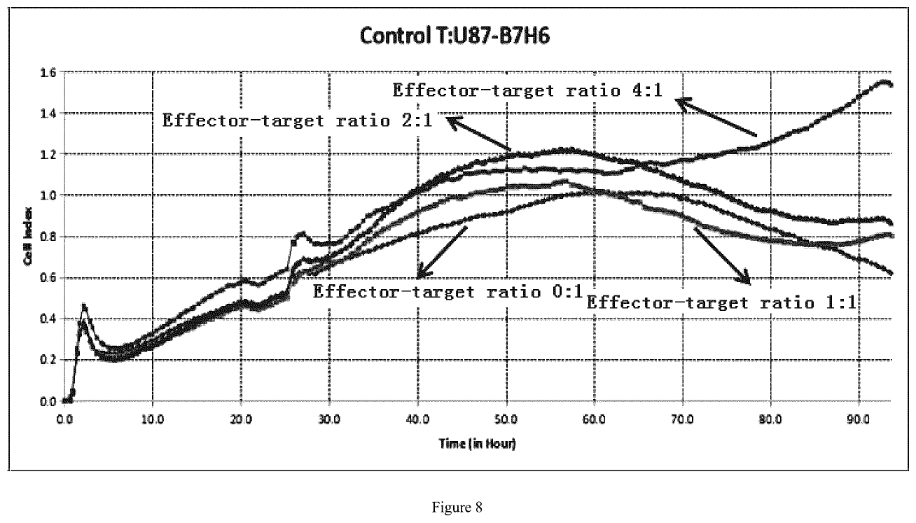

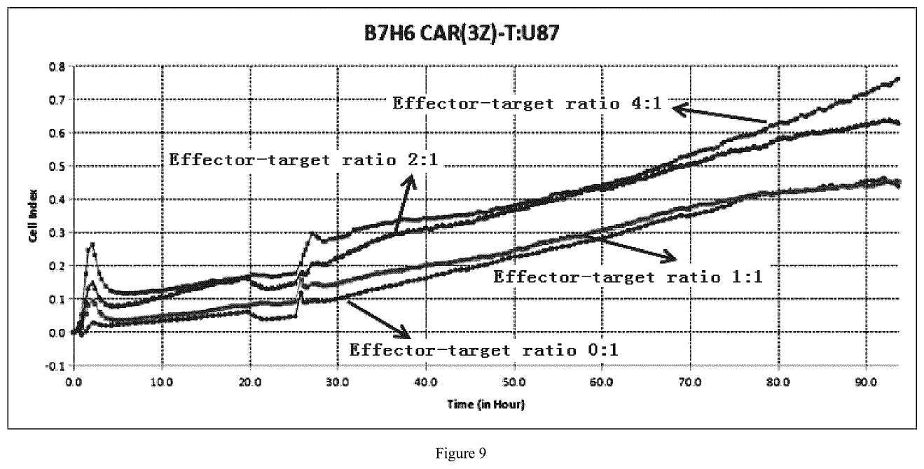

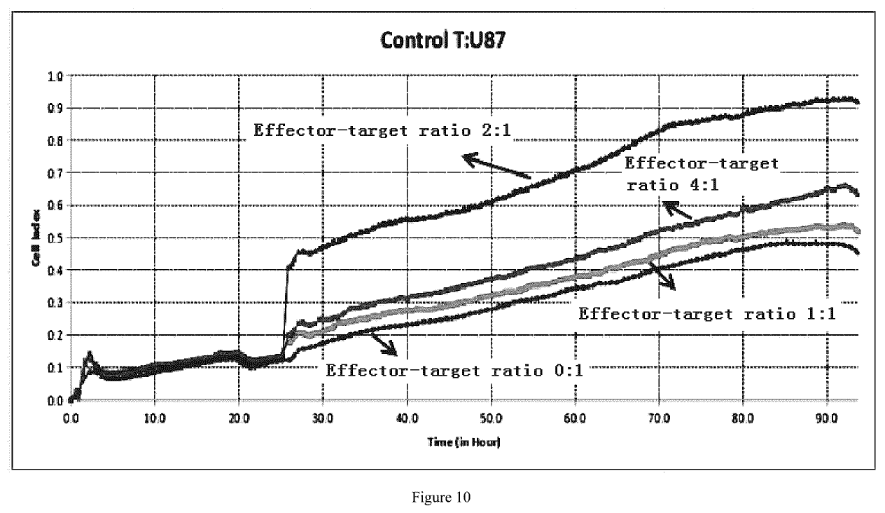

TABLE 4
Setups for effect target coculture experimental
groups for killing experiments

| | |
|---|---|
| Cell type | Target cells |

| | | |
|---|---|---|
| Effector cell | U87-B7H6(positive | U87(negative control |
| | | target cell) |
| CAR(CD3ζ1)-T | killing curve refers | — |
| | to FIG. 5 | |
| CAR(CD3ζ)-T | killing curve refers | killing curve refers |
| | to FIG. 6 | to FIG. 9 |
| CAR(Apd1)-T | killing curve refers | — |
| | to FIG. 7 | |
| Control T | killing curve refers | killing curve refers |
| | to FIG. 8 | to FIG. 10 |

Each effect target coculture experimental group is set with 4 effector-target ratio, including 0:1 (target cell blank control), 1:1, 2:1 and 4:1; two parallel experimental wells are set for each effective effector-target ratio, except for the target cell blank control having an effector-target ratio of 0:1. U87-B7H6 cells have 8 parallel wells, and U87 cells have 4 parallel wells. The average value is taken when analyzing the results.

The killing curves of the coculture experimental groups with various effector-target ratio are shown in FIG. 5-10. Time point 0.0 is the time point for seeding target cells, and effector cells are added to coculture the effector cells for about 26 hours. The entire experiment lasted for 96 hours. When effector T cells are added, it is 8 days after activation of CD3/CD28 magnetic beads, and 7 days after CAR virus transduction of T cells.

From the killing curve, it can be seen that for the positive target cell U87-B7H6, all three types of B7H6-CAR-T cells show significant killing effects, while the control T (Control T) cells do not show significant killing effects; for the negative control target cell U87, one of the three CAR-T cells tested, i.e. B7H6-CAR (CD3ζ1)-T and Control T do not show any killing effect.

From the killing curve, it can also be seen that the killing effect of the three types of CAR-T cells on the positive target cell U87-B7H6-eGFP is enhanced with the increase of the effector-target ratio (the slope of the curve increases) in the early stage of killing (approximately 12 hours after the start of coculture), but the killing effect tends to ease with the extension of coculture time.

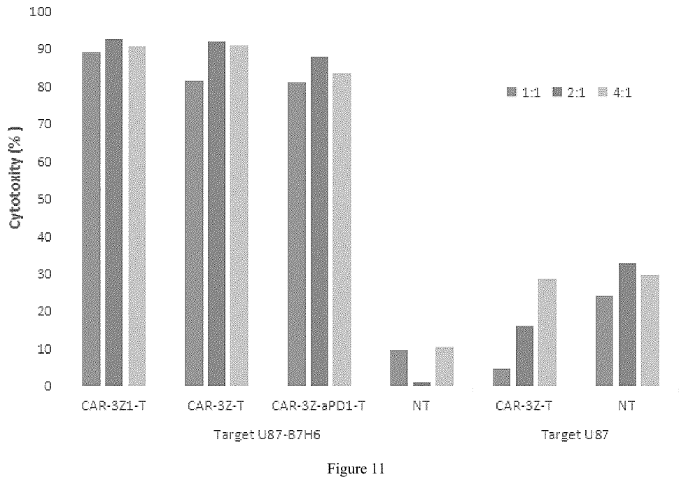

In order to further analyze the overall killing efficiency of B7H6-CAR-T on target cells, the cell index values at both ends of the early time period of coculture (i.e., the Cell Index values at 26:36:27 and 70:23:33) is intercepted to calculate the killing efficiency. The results are shown in Table 5 and FIG. 11.

TABLE 5
Killing efficiency of B7H6-CAR-T on target cell U87-B7H6/U87

| | Target cell | | | | | |
|---|---|---|---|---|---|---|
| | Target U87-B7H6 | | | | Target U87 | |
| | CAR | CAR | CAR | | CAR | |
| Effector T cell | (3ζ1)-T | (3ζ)-T | (aPD1)-T | Control T | (3ζ)-T | Control T |
| effector:target = 1:1 | 89.2 | 81.38 | 81.28 | 9.67 | 4.72 | 24.11 |
| effector:target = 2:1 | 92.59 | 92.0 | 88.06 | 0.95 | 16.2 | 32.82 |
| effector:target = 4:1 | 90.82 | 91.13 | 83.63 | 10.68 | 28.76 | 29.82 |

From the results in Table 5 and FIG. 11, it can be seen that:
1. Under the current CAR positivity rate, CAR-T in all three structures have extremely strong killing effects on target positive cells. However, at the investigated coculture endpoint, there is not much difference between the three effector-target ratio, wherein the killing effect is relatively strongest when the effector-target ratio is 2:1, while the killing effect of control T cells is extremely weak.
2. Among the three types of CAR-T cells, only CAR(3)ζ)-T represented the detection of its killing effect on the negative control target cell U87 and the results show that its killing effect increased with the increase of the effector-target ratio. The killing efficiency reached its highest at a effector-target ratio of 4:1, reaching 28.76%. However, compared with the positive target cell group, it is only 31.56% of its corresponding killing efficiency.
3. Control T cells have a certain non-target specific killing effect on negative control target cells, with the highest killing efficiency of 32.82% at an effective target ratio of 2:1, but it is only 35.45% of the killing efficiency for the experimental group with the strongest killing effect (CAR(3(1)-T: U87-B7H6 (E:T=2:1).

Based on the analysis of the comprehensive killing test curve and the killing efficiency chart, it can be concluded that the three types of B7H6-CAR-T cells have extremely strong and specific killing effects on B7H6 positive target cells. Although the present invention has been described with reference to exemplary embodiments, it should be understood that the present invention is not limited to the disclosed exemplary embodiments. Multiple adjustments or variations may be made to the exemplary embodiments of the present invention without departing from the scope or spirit of the present invention. The scope of claims should be based on the widest interpretation to cover all modifications and equivalent structures and functions.

1. An antibody or the antigen binding fragment thereof comprising a heavy chain variable region or a light chain variable region, wherein,
the heavy chain variable region comprises antigen complementary determining regions CDR1, CDR2, and CDR3 of the amino acid sequence shown in SEQ ID NO.: 11-13 respectively, and/or
the light chain variable region comprises antigen complementary determining regions CDR1, CDR2, and CDR3 of the amino acid sequence shown in SEQ ID NO.: 14-16 respectively; or
the heavy chain variable region comprises antigen complementary determining regions CDR1, CDR2, and CDR3 of the amino acid sequence shown in SEQ ID NO.: 17-19 respectively, and/or
the light chain variable region comprises antigen complementary determining regions CDR1, CDR2, and CDR3 of the amino acid sequence shown in SEQ ID NO.: 20-22 respectively.

2. The antibody or the antigen binding fragment thereof according to claim 1, wherein the antibody has any one of the amino acid sequences shown in (I), (II), or (III):
(I) the amino acid sequences obtained from the heavy chain variable region coding sequence shown in SEQ ID NO.: 23 and/or the light chain variable region coding sequence shown in SEQ ID NO.: 24; or
the amino acid sequences obtained from the heavy chain variable region coding sequence shown in SEQ ID NO.: 25 and/or the light chain variable region coding sequence shown in SEQ ID NO.: 26;
(II) the amino acid sequence having at least 90%, preferably at least 95%, further preferably at least 98%, and most preferably at least 99% homology with the amino acid sequence obtained from any of the encoding sequences shown in anyone of SEQ ID NO.: 23-26;
(III) the amino acid sequence obtained by modification, substitution, deletion or addition of one or more amino acids to the encoding sequence shown in anyone of SEQ ID NO.: 23-26.

3. The antibody or antigen binding fragment thereof according to claim 1, wherein the antibody comprises at least one of a monoclonal antibody, a chimeric antibody, a humanized antibody, or a bispecific antibody; the antigen binding fragments contain at least one of a Fab fragment, Fab′, a F(ab′)2 fragment, a single chain variable fragment scFv, a scFv-Fc fragment, or a single chain antibody ScAb.

4. A chimeric antigen receptor comprises:
1) recognizing the antigen binding domain of B7H6 antigen, wherein the antigen binding domain comprises the antibody or the antigen binding fragment thereof according to claim 1;
2) a transmembrane domain; and
3) an intracellular signal transduction domain;
preferably, the chimeric antigen receptor further comprises a hinge area;
preferably, the chimeric antigen receptor further comprises a suicide switch molecule;
preferably, the chimeric antigen receptor further comprises an intracellular costimulatory domains;
preferably, the transmembrane domain is selected from at least one peptides of CD28, NKp30, CDS, DAP10, 4-1BB, DAP12, CD3C, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1, ICOS(CD278), 4-1BB(CD137), GITR, CD40, BAFFR, HVEM(LIGHT), SLAMF7, NKp80(KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4 (CD244, 2B4), CD84, CD96, CEACAM1, CRTAM, Ly9(CD229), CD160(BY55), PSGL1, CD100(SEMA4D), SLAMF6(NTB-A, Ly108), SLAM(SLAMF1, CD150, IPO-3), BLAME(SLAMF8), SELPLG(CD162), LTBR, PAG/Cbp or any combinations thereof;
preferably, the intracellular signal transduction domain is selected from at least one of CD8, CD3ζ, CD36, CD3γ, CD3ε, FcγRI-γ, FcγRIII-γ, FcεRIβ, FcεRIγ, DAP10, DAP12, CD32, B7H69a, B7H69b, CD28, CD3C, CD4, b2c, CD137 (4-1BB), ICOS, CD27, CD28 δ, CD80, NKp30, OX40 or the combination thereof;
preferably, the intracellular signaling domain comprises a shortened CD3C chain retaining at least one ITAM motif of the CD3C chain, preferably retaining the first ITAM motif among the three ITAMs in the CD3C chain.

5. The chimeric antigen receptor according to claim 4, wherein it further comprises a fusion fragment comprising a cytokine and an anti-PD1-scFv or PD1 antigen binding fragment;
preferably, the cytokine comprises IL21.

6. A separated nucleic acid molecule encoding the antibody or the antigen binding fragment thereof according to claim 1.

7. A carrier comprising the nucleic acid molecule according to claim 6.

8. A host cell comprising the carrier according to claim 7.

9. An immunologic effector cell expressing the antibody or the antigen binding fragment thereof according to claim 1, wherein
the immunologic effector cells are selected from at least one of a leukocyte, a monocyte, a macrophage, a dendritic cell, a mast cell, a neutrophil, a basophil, an eosinophil, a αβ T cell, a γδ T cell, a natural killer (NK) cell, a natural killer T (NKT) cell, a B cell, a natural lymphoid cell (ILC), a cytokine induced killer (CIK) cell, a cytotoxic T lymphocyte (CTL), a lymphokine activated killer (LAK) cell, a T lymphocyte, a peripheral blood mononuclear cell and a hematopoietic stem cell or any combinations thereof.

10. An application of a reagent in the preparation of drugs for preventing and/or treating cancer or tumors, wherein the reagent includes the antibody or the antigen binding fragment thereof according to claim 1;
preferably, the cancer or tumor includes cancer or tumor associated with B7H6 expression;
preferably, the cancer or tumor includes myeloid leukemia, acute non lymphocytic leukemia, acute lymphoblastic leukemia, B-cell lymphoma, breast cancer, cervical cancer, clear cell renal cell cancer, dermatofibrosarcoma protuberans, gastric sarcoma, gastrointestinal stromal tumor, glioblastoma, leiomyosarcoma, invasive ductal breast cancer, malignant fibrous histiocytoma, melanoma, ovarian serous surface papillary cancer, pancreatic cancer, prostate cancer, T-cell acute lymphocytic leukemia, small cell lung cancer or T-cell lymphoma;
preferably, the application further includes the application of the antibody or the antigen binding fragment thereof according to claim 1;
preferably, the other drugs include diagnostic agents, prophylactic agents, and/or therapeutic agents;
preferably, the other drugs include CD20 targeting antibody drugs.
